(19) United States
(12) Patent Application Publication
(10) Pub. No.: US 2013/0303763 A1
(43) Pub. Date: Nov. 14, 2013

(54) METHODS AND COMPOSITIONS FOR THE TREATMENT OF NECROTIZING ENTEROCOLITIS

(71) Applicants: Michael D. GERSHON, New York, NY (US); Kara Gross MARGOLIS, New York, NY (US)

(72) Inventors: Michael D. GERSHON, New York, NY (US); Kara Gross MARGOLIS, New York, NY (US)

(21) Appl. No.: 13/852,327

(22) Filed: Mar. 28, 2013

Related U.S. Application Data
(60) Provisional application No. 61/617,910, filed on Mar. 30, 2012.

(51) Int. Cl. C07D 239/34; C07D 403/12

(52) U.S. Cl. CPC C07D 239/34; C07D 403/12
USPC 544/319

(57) ABSTRACT

This invention relates to tryptophan hydroxylase inhibitors, compositions comprising them, and methods of their use for the treatment, management and/or prevention of necrotizing enterocolitis.

This application claims priority to U.S. provisional patent application No. 61/617,910, filed Mar. 30, 2012, the entirety of which is incorporated herein by reference.

### 1. FIELD OF THE INVENTION

This invention relates to tryptophan hydroxylase inhibitors, compositions comprising them, and methods of their use for the treatment, management and/or prevention of necrotizing enterocolitis.

### 2. BACKGROUND

The neurotransmitter serotonin [5-hydroxytryptamine (5-HT)] is involved in multiple central nervous facets of mood control and in regulating sleep, anxiety, alcoholism, drug abuse, food intake, and sexual behavior. In peripheral tissues, serotonin is implicated in the regulation of vascular tone, gut motility, primary hemostasis, and cell-mediated immune responses. See, e.g., Walther, D. J., et al., Science 299:76 (2003); Chen, J. J., et al., J. Neuroscience 21(16):6348-6361 (2001).

The enzyme tryptophan hydroxylase (TPH) catalyzes the rate limiting step of the biosynthesis of serotonin. Two isoforms of TPH have been reported: TPH1, which is expressed in the periphery, primarily in the gastrointestinal (GI) tract; and TPH2, which is expressed in the brain. Id. The isoform TPH1 is encoded by the tph1 gene; TPH2 is encoded by the tph2 gene. Id. Small molecule inhibitors of TPH—and TPH1 in particular—have been disclosed for the treatment of a number of disease and disorders. See, e.g., U.S. Pat. Nos. 8,093,291; 8,063,057; 7,968,559; 7,875,622; 7,553,840; 7,723,345; and 7,709,493. The TPH1 inhibitors LX1031 and LX1033 (Lexicon Pharmaceuticals, Inc.) have been studied in human clinical trials for the treatment of diarrhea-predominant irritable bowel syndrome. The TPH1 inhibitor LX1032 (Lexicon Pharmaceuticals, Inc.) has been studied in human clinical trials for the treatment of carcinoid syndrome.

Necrotizing enterocolitis (NEC) is a devastating inflammatory gastrointestinal disease that predominately occurs in premature infants. In its most severe form, it is characterized by extensive hemorrhagic inflammatory necrosis of the distal ileum and proximal colon. The disease reportedly affects 0.3-2.4 infants per 1000 live births: seven to 11 percent of infants weighing less than 1.5 kg. Mortality occurs in 10-50 percent of affected infants. Unfortunately, there are few methods of treating the disease, and its etiology is unclear. It is known that prematurity, bacterial colonization, intestinal hypoxia-ischemia and formula feeding contribute to its pathogenesis. Thus, the disease is typically addressed by intravenous administration of antibiotics combined with no oral feeding, and, in more severe cases, by surgical resection of the affected bowel.

### 3. SUMMARY OF THE INVENTION

This invention is directed, in part, to compositions and methods for the treatment, management, and/or prevention of necrotizing enterocolitis (NEC).

One embodiment of the invention encompasses a method of treating, managing or preventing NEC that comprises administering to that patient a therapeutically or prophylactically effective amount of a tryptophan hydroxylase (TPH) inhibitor. Particular TPH inhibitors include compounds of the formula:

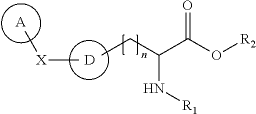

and pharmaceutically acceptable salts thereof, wherein: A is optionally substituted cycloalkyl, aryl, or heterocycle; X is a bond (i.e., A is directly bound to D), —O—, —S—, —C(O)—, —C(R₄)═, ═C(R₄)—, —C(R₃R₄)—, —C(R₄)═C(R₄)—, —C≡C—, —N(R₅)—, —N(R₅)C(O)N(R₅)—, —C(R₃R₄)N(R₅)—, —N(R₅)C(R₃R₄)—, —ONC(R₃)—, —C(R₃)NO—, —C(R₃R₄)O—, —OC(R₃R₄)—, —S(O₂)—, —S(O₂)N(R₅)—, —N(R₅)S(O₂)—, —C(R₃R₄)S(O₂)—, or —S(O₂)C(R₃R₄)—; D is optionally substituted aryl or heterocycle; R₁ is hydrogen or optionally substituted alkyl, alkyl-aryl, alkyl-heterocycle, aryl, or heterocycle; R₂ is hydrogen or optionally substituted alkyl, alkyl-aryl, alkyl-heterocycle, aryl, or heterocycle; R₃ is hydrogen, alkoxy, amino, cyano, halogen, hydroxyl, or optionally substituted alkyl; R₄ is hydrogen, alkoxy, amino, cyano, halogen, hydroxyl, or optionally substituted alkyl or aryl; each R₅ is independently hydrogen or optionally substituted alkyl or aryl; and n is 0-3.

Another embodiment encompasses pharmaceutical compositions for the treatment of NEC comprising a TPH inhibitor, optionally in combination with one or more additional drugs.

### 4. BRIEF DESCRIPTION OF THE FIGURES

FIG. 1 shows the effect of administring 100 or 200 mg/kg of (2S)-ethyl 2-amino-3-(4-(2-amino-6-(2,2,2-trifluoro-1-(3′-fluoro-[1,1′-biphenyl]-4-yl)ethoxy)pyrimidin-4-yl)phenyl)propanoate (Compound), as compared to vehicle control, on the weight of mice suffering from NEC.

FIG. 2 shows the effect of administring 100 or 200 mg/kg of (2S)-ethyl 2-amino-3-(4-(2-amino-6-(2,2,2-trifluoro-1-(3′-fluoro-[1,1′-biphenyl]-4-yl)ethoxy)pyrimidin-4-yl)phenyl)propanoate (Compound), as compared to vehicle control, on the survival of mice suffering from NEC.

### 5. DETAILED DESCRIPTION

This invention is based on the discovery—using SERT knockout mice and TPH1 knockout mice—that serotonin plays an important role in the severity of NEC. It is further based on the discovery that the oral administration of TPH1 inhibitors can reduce the severity and mortality of NEC in mice.

### 5.1. Definitions

Unless otherwise indicated, the term “alkenyl” means a straight chain, branched and/or cyclic hydrocarbon having from 2 to 20 (e.g., 2 to 10 or 2 to 6) carbon atoms, and including at least one carbon-carbon double bond. Representative alkenyl moieties include vinyl, allyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, 1-hexenyl, 2-hexenyl, 3-hexenyl, 1-heptenyl, 2-heptenyl, 3-heptenyl, 1-octenyl, 2-octenyl, 3-octenyl, 1-nonenyl, 2-nonenyl, 3-nonenyl, 1-decenyl, 2-decenyl and 3-decenyl.

Unless otherwise indicated, the term “alkyl” means a straight chain, branched and/or cyclic (“cycloalkyl”) hydrocarbon having from 1 to 20 (e.g., 1 to 10 or 1 to 4) carbon atoms. Alkyl moieties having from 1 to 4 carbons are referred to as “lower alkyl.” Examples of alkyl groups include methyl, ethyl, propyl, isopropyl, n-butyl, t-butyl, isobutyl, pentyl, hexyl, isohexyl, heptyl, 4,4-dimethylpentyl, octyl, 2,2,4-trimethylpentyl, nonyl, decyl, undecyl and dodecyl. Cycloalkyl moieties may be monocyclic or multicyclic, and examples include cyclopropyl, cyclobutyl, cyclopentyl, cyclohexyl, and adamantyl. Additional examples of alkyl moieties have linear, branched and/or cyclic portions (e.g., 1-ethyl-4-methyl-cyclohexyl). The term “alkyl” includes saturated hydrocarbons as well as alkenyl and alkynyl moieties.

Unless otherwise indicated, the term “alkoxy” means an —O-alkyl group. Examples of alkoxy groups include —OCH₃, —OCH₂CH₃, —O(CH₂)₂CH₃, —O(CH₂)₃CH₃, —O(CH₂)₄CH₃, and —O(CH₂)₅CH₃.

Unless otherwise indicated, the term “alkylaryl” or “alkyl-aryl” means an alkyl moiety bound to an aryl moiety.

Unless otherwise indicated, the term “alkylheteroaryl” or “alkyl-heteroaryl” means an alkyl moiety bound to a heteroaryl moiety.

Unless otherwise indicated, the term “alkylheterocycle” or “alkyl-heterocycle” means an alkyl moiety bound to a heterocycle moiety.

Unless otherwise indicated, the term “alkynyl” means a straight chain, branched or cyclic hydrocarbon having from 2 to 20 (e.g., 2 to 20 or 2 to 6) carbon atoms, and including at least one carbon-carbon triple bond. Representative alkynyl moieties include acetylenyl, propynyl, 1-butynyl, 2-butynyl, 1-pentynyl, 2-pentynyl, 3-methyl-1-butynyl, 4-pentynyl, 1-hexynyl, 2-hexynyl, 5-hexynyl, 1-heptynyl, 2-heptynyl, 6-heptynyl, 1-octynyl, 2-octynyl, 7-octynyl, 1-nonynyl, 2-nonynyl, 8-nonynyl, 1-decynyl, 2-decynyl and 9-decynyl.

Unless otherwise indicated, the term “aryl” means an aromatic ring or an aromatic or partially aromatic ring system composed of carbon and hydrogen atoms. An aryl moiety may comprise multiple rings bound or fused together. Examples of aryl moieties include anthracenyl, azulenyl, biphenyl, fluorenyl, indan, indenyl, naphthyl, phenanthrenyl, phenyl, 1,2,3,4-tetrahydro-naphthalene, and tolyl.

Unless otherwise indicated, the term “arylalkyl” or “aryl-alkyl” means an aryl moiety bound to an alkyl moiety.

Unless otherwise indicated, the terms “biohydrolyzable amide,” “biohydrolyzable ester,” “biohydrolyzable carbamate,” “biohydrolyzable carbonate,” “biohydrolyzable ureido” and “biohydrolyzable phosphate” mean an amide, ester, carbamate, carbonate, ureido, or phosphate, respectively, of a compound that either: 1) does not interfere with the biological activity of the compound but can confer upon that compound advantageous properties in vivo, such as uptake, duration of action, or onset of action; or 2) is biologically inactive but is converted in vivo to the biologically active compound. Examples of biohydrolyzable esters include lower alkyl esters, alkoxyacyloxy esters, alkyl acylamino alkyl esters, and choline esters. Examples of biohydrolyzable amides include lower alkyl amides, α-amino acid amides, alkoxyacyl amides, and alkylaminoalkyl-carbonyl amides. Examples of biohydrolyzable carbamates include lower alkylamines, substituted ethylenediamines, aminoacids, hydroxyalkylamines, heterocyclic and heteroaromatic amines, and polyether amines.

Unless otherwise indicated, the terms “halogen” and “halo” encompass fluorine, chlorine, bromine, and iodine.

Unless otherwise indicated, the term “heteroalkyl” refers to an alkyl moiety (e.g., linear, branched or cyclic) in which at least one of its carbon atoms has been replaced with a heteroatom (e.g., N, O or S).

Unless otherwise indicated, the term “heteroaryl” means an aryl moiety wherein at least one of its carbon atoms has been replaced with a heteroatom (e.g., N, O or S). Examples include acridinyl, benzimidazolyl, benzofuranyl, benzoisothiazolyl, benzoisoxazolyl, benzoquinazolinyl, benzothiazolyl, benzoxazolyl, furyl, imidazolyl, indolyl, isothiazolyl, isoxazolyl, oxadiazolyl, oxazolyl, phthalazinyl, pyrazinyl, pyrazolyl, pyridazinyl, pyridyl, pyrimidinyl, pyrimidyl, pyrrolyl, quinazolinyl, quinolinyl, tetrazolyl, thiazolyl, and triazinyl.

Unless otherwise indicated, the term “heteroarylalkyl” or “heteroaryl-alkyl” means a heteroaryl moiety bound to an alkyl moiety.

Unless otherwise indicated, the term “heterocycle” refers to an aromatic, partially aromatic or non-aromatic monocyclic or polycyclic ring or ring system comprised of carbon, hydrogen and at least one heteroatom (e.g., N, O or S). A heterocycle may comprise multiple (i.e., two or more) rings fused or bound together. Heterocycles include heteroaryls. Examples include benzo[1,3]dioxolyl, 2,3-dihydro-benzo[1,4]dioxinyl, cinnolinyl, furanyl, hydantoinyl, morpholinyl, oxetanyl, oxiranyl, piperazinyl, piperidinyl, pyrrolidinonyl, pyrrolidinyl, tetrahydrofuranyl, tetrahydropyranyl, tetrahydropyridinyl, tetrahydropyrimidinyl, tetrahydrothiophenyl, tetrahydrothiopyranyl and valerolactamyl.

Unless otherwise indicated, the term “heterocyclealkyl” or “heterocycle-alkyl” refers to a heterocycle moiety bound to an alkyl moiety.

Unless otherwise indicated, the term “heterocycloalkyl” refers to a non-aromatic heterocycle.

Unless otherwise indicated, the term “heterocycloalkylalkyl” or “heterocycloalkyl-alkyl” refers to a heterocycloalkyl moiety bound to an alkyl moiety.

Unless otherwise indicated, the terms “manage,” “managing” and “management” encompass preventing the recurrence of the specified disease or disorder, or of one or more of its symptoms, in a patient who has already suffered from the disease or disorder, and/or lengthening the time that a patient who has suffered from the disease or disorder remains in remission. The terms encompass modulating the threshold, development and/or duration of the disease or disorder, or changing the way that a patient responds to the disease or disorder.

Unless otherwise indicated, the term “pharmaceutically acceptable salts” refers to salts prepared from pharmaceutically acceptable non-toxic acids or bases including inorganic acids and bases and organic acids and bases. Suitable pharmaceutically acceptable base addition salts include metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine) and procaine. Suitable non-toxic acids include inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Specific non-toxic acids include hydrochloric, hydrobromic, phosphoric, sulfuric, and methanesulfonic acids. Examples of specific salts thus include hydrochloride and mesylate salts. Others are well-known in the art. See, e.g., Remington's Pharmaceutical Sciences, 18th ed. (Mack Publishing, Easton Pa.: 1990) and Remington: The Science and Practice of Pharmacy, 19th ed. (Mack Publishing, Easton Pa.: 1995).

Unless otherwise indicated, the term “potent TPH1 inhibitor” is a compound that has a TPH1_IC₅₀ of less than about 10 μM.

Unless otherwise indicated, the terms “prevent,” “preventing” and “prevention” contemplate an action that occurs before a patient begins to suffer from the specified disease or disorder, which inhibits or reduces the severity of the disease or disorder, or of one or more of its symptoms. The terms encompass prophylaxis.

Unless otherwise indicated, the term “prodrug” encompasses pharmaceutically acceptable esters, carbonates, thiocarbonates, N-acyl derivatives, N-acyloxyalkyl derivatives, quaternary derivatives of tertiary amines, N-Mannich bases, Schiff bases, amino acid conjugates, phosphate esters, metal salts and sulfonate esters of compounds disclosed herein. Examples of prodrugs include compounds that comprise a biohydrolyzable moiety (e.g., a biohydrolyzable amide, biohydrolyzable carbamate, biohydrolyzable carbonate, biohydrolyzable ester, biohydrolyzable phosphate, or biohydrolyzable ureide analog). Prodrugs of compounds disclosed herein are readily envisioned and prepared by those of ordinary skill in the art. See, e.g., Design of Prodrugs, Bundgaard, A. Ed., Elseview, 1985; Bundgaard, H., “Design and Application of Prodrugs,” A Textbook of Drug Design and Development, Krosgaard-Larsen and H. Bundgaard, Ed., 1991, Chapter 5, p. 113-191; and Bundgaard, H., Advanced Drug Delivery Review, 1992, 8, 1-38.

Unless otherwise indicated, a “prophylactically effective amount” of a compound is an amount sufficient to prevent a disease or condition, or one or more symptoms associated with the disease or condition, or prevent its recurrence. A prophylactically effective amount of a compound is an amount of therapeutic agent, alone or in combination with other agents, which provides a prophylactic benefit in the prevention of the disease. The term “prophylactically effective amount” can encompass an amount that improves overall prophylaxis or enhances the prophylactic efficacy of another prophylactic agent.

Unless otherwise indicated, the term “protecting group” or “protective group,” when used to refer to part of a molecule subjected to a chemical reaction, means a chemical moiety that is not reactive under the conditions of that chemical reaction, and which may be removed to provide a moiety that is reactive under those conditions. Protecting groups are well known in the art. See, e.g., Greene, T. W. and Wuts, P. G. M., Protective Groups in Organic Synthesis (3^{rd} ed., John Wiley & Sons: 1999); Larock, R. C., Comprehensive Organic Transformations (2^{nd} ed., John Wiley & Sons: 1999). Some examples include benzyl, diphenylmethyl, trityl, Cbz, Boc, Fmoc, methoxycarbonyl, ethoxycarbonyl, and pthalimido.

Unless otherwise indicated, the term “selective TPH1 inhibitor” is a compound that has a TPH2_IC₅₀ that is at least about 10 times greater than its TPH1_IC₅₀.

Unless otherwise indicated, the term “stereomerically enriched composition of” a compound refers to a mixture of the named compound and its stereoisomer(s) that contains more of the named compound than its stereoisomer(s). For example, a stereoisomerically enriched composition of (S)-butan-2-ol encompasses mixtures of (S)-butan-2-ol and (R)-butan-2-ol in ratios of, e.g., about 60/40, 70/30, 80/20, 90/10, 95/5, and 98/2.

Unless otherwise indicated, the term “stereoisomeric mixture” encompasses racemic mixtures as well as stereomerically enriched mixtures (e.g., R/S=30/70, 35/65, 40/60, 45/55, 55/45, 60/40, 65/35 and 70/30).

Unless otherwise indicated, the term “stereomerically pure” means a composition that comprises one stereoisomer of a compound and is substantially free of other stereoisomers of that compound. For example, a stereomerically pure composition of a compound having one stereocenter will be substantially free of the opposite stereoisomer of the compound. A stereomerically pure composition of a compound having two stereocenters will be substantially free of other diastereomers of the compound. A typical stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, greater than about 97% by weight of one stereoisomer of the compound and less than about 3% by weight of the other stereoisomers of the compound, or greater than about 99% by weight of one stereoisomer of the compound and less than about 1% by weight of the other stereoisomers of the compound.

Unless otherwise indicated, the term “substituted,” when used to describe a chemical structure or moiety, refers to a derivative of that structure or moiety wherein one or more of its hydrogen atoms is substituted with an atom, chemical moiety or functional group such as, but not limited to, alcohol, aldehylde, alkoxy, alkanoyloxy, alkoxycarbonyl, alkenyl, alkyl (e.g., methyl, ethyl, propyl, t-butyl), alkynyl, alkylcarbonyloxy (—OC(O)alkyl), amide (—C(O)NH-alkyl- or -alkylNHC(O)alkyl), amidinyl (—C(NH)NH-alkyl or —C(NR)NH₂), amine (primary, secondary and tertiary such as alkylamino, arylamino, arylalkylamino), aroyl, aryl, aryloxy, azo, carbamoyl (—NHC(O)O-alkyl- or —OC(O)NH-alkyl), carbamyl (e.g., CONH₂, as well as CONH-alkyl, CONH-aryl, and CONH-arylalkyl), carbonyl, carboxyl, carboxylic acid, carboxylic acid anhydride, carboxylic acid chloride, cyano, ester, epoxide, ether (e.g., methoxy, ethoxy), guanidino, halo, haloalkyl (e.g., —CCl₃, —CF₃, —C(CF₃)₃), heteroalkyl, hemiacetal, imine (primary and secondary), isocyanate, isothiocyanate, ketone, nitrile, nitro, oxygen (i.e., to provide an oxo group), phosphodiester, sulfide, sulfonamido (e.g., SO₂NH₂), sulfone, sulfonyl (including alkylsulfonyl, arylsulfonyl and arylalkylsulfonyl), sulfoxide, thiol (e.g., sulfhydryl, thioether) and urea (—NHCONH-alkyl-).

Unless otherwise indicated, a “therapeutically effective amount” of a compound is an amount sufficient to provide a therapeutic benefit in the treatment or management of a disease or condition, or to delay or minimize one or more symptoms associated with the disease or condition. A therapeutically effective amount of a compound is an amount of therapeutic agent, alone or in combination with other therapies, which provides a therapeutic benefit in the treatment or management of the disease or condition. The term “therapeutically effective amount” can encompass an amount that improves overall therapy, reduces or avoids symptoms or causes of a disease or condition, or enhances the therapeutic efficacy of another therapeutic agent.

Unless otherwise indicated, the terms “treat,” “treating” and “treatment” contemplate an action that occurs while a patient is suffering from the specified disease or disorder, which reduces the severity of the disease or disorder, or one or more of its symptoms, or retards or slows the progression of the disease or disorder.

Unless otherwise indicated, the term “include” has the same meaning as “include” and the term “includes” has the same meaning as “includes, but is not limited to.” Similarly, the term “such as” has the same meaning as the term “such as, but not limited to.”

Unless otherwise indicated, one or more adjectives immediately preceding a series of nouns is to be construed as applying to each of the nouns. For example, the phrase “optionally substituted alky, aryl, or heteroaryl” has the same meaning as “optionally substituted alky, optionally substituted aryl, or optionally substituted heteroaryl.”

It should be noted that a chemical moiety that forms part of a larger compound may be described herein using a name commonly accorded it when it exists as a single molecule or a name commonly accorded its radical. For example, the terms “pyridine” and “pyridyl” are accorded the same meaning when used to describe a moiety attached to other chemical moieties. Thus, the two phrases “XOH, wherein X is pyridyl” and “XOH, wherein X is pyridine” are accorded the same meaning, and encompass the compounds pyridin-2-ol, pyridin-3-ol and pyridin-4-ol.

It should also be noted that if the stereochemistry of a structure or a portion of a structure is not indicated with, for example, bold or dashed lines, the structure or the portion of the structure is to be interpreted as encompassing all stereoisomers of it. Similarly, names of compounds having one or more chiral centers that do not specify the stereochemistry of those centers encompass pure stereoisomers and mixtures thereof. Moreover, any atom shown in a drawing with unsatisfied valences is assumed to be attached to enough hydrogen atoms to satisfy the valences. In addition, chemical bonds depicted with one solid line parallel to one dashed line encompass both single and double (e.g., aromatic) bonds, if valences permit. This invention encompasses tautomers and solvates (e.g., hydrates) of the compounds disclosed herein.

### 5.2. Compounds

Methods and compositions of this invention utilize TPH inhibitors, examples of which are disclosed in U.S. Pat. Nos. 8,093,291; 8,063,057; 7,968,559; 7,875,622; 7,553,840; 7,723,345; and 7,709,493.

Particular TPH inhibitors are compounds of the formula:

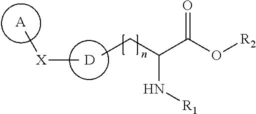

and pharmaceutically acceptable salts thereof, wherein: A is optionally substituted cycloalkyl, aryl, or heterocycle; X is a bond, —O—, —S—, —C(O)—, —C(R₄)═, ═C(R₄)—, —C(R₃R₄)—, —C(R₄)═C(R₄)—, —C≡C—, —N(R₅)—, —N(R₅)C(O)N(R₅)—, —C(R₃R₄)N(R₅)—, —N(R₅)C(R₃R₄)—, —ONC(R₃)—, —C(R₃)NO—, —C(R₃R₄)O—, —OC(R₃R₄)—, —S(O₂)—, —S(O₂)N(R₅)—, —N(R₅)S(O₂)—, —C(R₃R₄)S(O₂)—, or —S(O₂)C(R₃R₄)—; D is optionally substituted aryl or heterocycle; R₁ is hydrogen or optionally substituted alkyl, alkyl-aryl, alkyl-heterocycle, aryl, or heterocycle; R₂ is hydrogen or optionally substituted alkyl, alkyl-aryl, alkyl-heterocycle, aryl, or heterocycle; R₃ is hydrogen, alkoxy, amino, cyano, halogen, hydroxyl, or optionally substituted alkyl; R₄ is hydrogen, alkoxy, amino, cyano, halogen, hydroxyl, or optionally substituted alkyl or aryl; each R₅ is independently hydrogen or optionally substituted alkyl or aryl; and n is 0-3.

Particular compounds are of the formula:

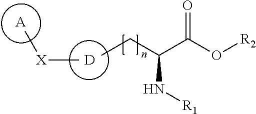

Also encompassed by the invention are compounds of the formula:

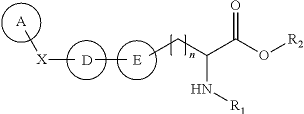

and pharmaceutically acceptable salts thereof, wherein: A is optionally substituted cycloalkyl, aryl, or heterocycle; X is a bond, —O—, —S—, —C(O)—, —C(R₄)═, ═C(R₄)—, —C(R₃R₄)—, —C(R₄)═C(R₄)—, —C≡C—, —N(R₅)—, —N(R₅)C(O)N(R₅)—, —C(R₃R₄)N(R₅)—, —N(R₅)C(R₃R₄)—, —ONC(R₃)—, —C(R₃)NO—, —C(R₃R₄)O—, —OC(R₃R₄)—, —S(O₂)—, —S(O₂)N(R₅)—, —N(R₅)S(O₂)—, —C(R₃R₄)S(O₂)—, or —S(O₂)C(R₃R₄)—; D is optionally substituted aryl or heterocycle; E is optionally substituted aryl or heterocycle; R₁ is hydrogen or optionally substituted alkyl, alkyl-aryl, alkyl-heterocycle, aryl, or heterocycle; R₂ is hydrogen or optionally substituted alkyl, alkyl-aryl, alkyl-heterocycle, aryl, or heterocycle; R₃ is hydrogen, alkoxy, amino, cyano, halogen, hydroxyl, or optionally substituted alkyl; R₄ is hydrogen, alkoxy, amino, cyano, halogen, hydroxyl, or optionally substituted alkyl or aryl; R₅ is hydrogen or optionally substituted alkyl or aryl; and n is 0-3.

Particular compounds are of the formula:

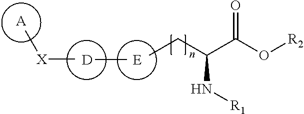

With regard to the formulae disclosed herein, particular compounds include those wherein A is optionally substituted cycloalkyl (e.g., 6-membered and 5-membered). In some, A is optionally substituted aryl (e.g., phenyl or naphthyl). In others, A is optionally substituted heterocycle (e.g., 6-membered and 5-membered). Examples of 6-membered heterocycles include pyridine, pyridazine, pyrimidine, pyrazine, and triazine. Examples of 5-membered heterocycles include pyrrole, imidazole, triazole, thiazole, thiophene, and furan. In some compounds, A is aromatic. In others, A is not aromatic. In some, A is an optionally substituted bicyclic moiety (e.g., indole, iso-indole, pyrrolo-pyridine, or napthylene).

Particular compounds are of the formula:

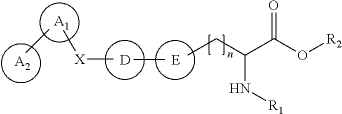

wherein: each of A₁ and A₂ is independently a monocyclic optionally substituted cycloalkyl, aryl, or heterocycle. Compounds encompassed by this formula include those wherein A₁ and/or A₂ is optionally substituted cycloalkyl (e.g., 6-membered and 5-membered). In some, A₁ and/or A₂ is optionally substituted aryl (e.g., phenyl or naphthyl). In others, A₁ and/or A₂ is optionally substituted heterocycle (e.g., 6-membered and 5-membered). Examples of 6-membered heterocycles include pyridine, pyridazine, pyrimidine, pyrazine, and triazine. Examples of 5-membered heterocycles include pyrrole, imidazole, triazole, thiazole, thiophene, and furan. In some compounds, A₁ and/or A₂ is aromatic. In others, A₁ and/or A₂ is not aromatic.

With regard to the formulae disclosed herein, particular compounds include those wherein D is optionally substituted aryl (e.g., phenyl or naphthyl). In others, D is optionally substituted heterocycle (e.g., 6-membered and 5-membered). Examples of 6-membered heterocycles include pyridine, pyridazine, pyrimidine, pyrazine, and triazine. Examples of 5-membered heterocycles include pyrrole, imidazole, triazole, thiazole, thiophene, and furan. In some compounds, D is aromatic. In others, D is not aromatic. In some, D is an optionally substituted bicyclic moiety (e.g., indole, iso-indole, pyrrolo-pyridine, or napthylene).

With regard to the various formulae disclosed herein, particular compounds include those wherein E is optionally substituted aryl (e.g., phenyl or naphthyl). In others, E is optionally substituted heterocycle (e.g., 6-membered and 5-membered). Examples of 6-membered heterocycles include pyridine, pyridazine, pyrimidine, pyrazine, and triazine. Examples of 5-membered heterocycles include pyrrole, imidazole, triazole, thiazole, thiophene, and furan. In some compounds, E is aromatic. In others, E is not aromatic. In some, E is an optionally substituted bicyclic moiety (e.g., indole, iso-indole, pyrrolo-pyridine, or napthylene).

With regard to the various formulae disclosed herein, particular compounds include those wherein R₁ is hydrogen or optionally substituted alkyl.

In some, R₂ is hydrogen or optionally substituted alkyl.

In some, n is 1 or 2.

In some, X is a bond or S. In others, X is —C(R₄)═, ═C(R₄)—, —C(R₃R₄)—, —C(R₄)═C(R₄)—, or —C≡C—, and, for example, R₄ is independently hydrogen or optionally substituted alkyl. In others, X is —O—, —C(R₃R₄)O—, or —OC(R₃R₄)—, and, for example, R₃ is hydrogen or optionally substituted alkyl, and R₄ is hydrogen or optionally substituted alkyl. In some, R₃ is hydrogen and R₄ is trifluoromethyl. In some compounds, X is —S(O₂)—, —S(O₂)N(R₅)—, —N(R₅)S(O₂)—, —C(R₃R₄)S(O₂)—, or —S(O₂)C(R₃R₄)—, and, for example, R₃ is hydrogen or optionally substituted alkyl, R₄ is hydrogen or optionally substituted alkyl, and R₅ is hydrogen or optionally substituted alkyl. In others, X is —N(R₅)—, —N(R₅)C(O)N(R₅)—, —C(R₃R₄)N(R₅)—, or —N(R₅)C(R₃R₄)—, and, for example, R₃ is hydrogen or optionally substituted alkyl, R₄ is hydrogen or optionally substituted alkyl, and each R₅ is independently hydrogen or optionally substituted alkyl.

Some compounds of the invention are encompassed by the formula:

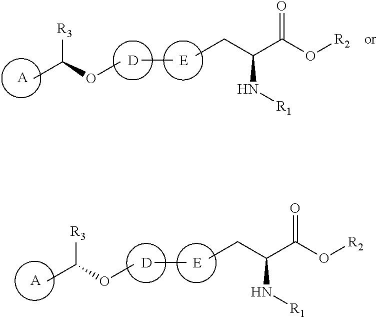

wherein, for example, R₃ is trifluoromethyl. Others are encompassed by the formula:

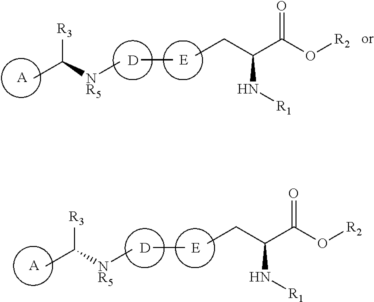

wherein, for example, R₃ is hydrogen.

Some compounds are encompassed by the formula:

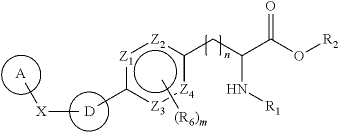

wherein: each of Z₁, Z₂, Z₃, and Z₄ is independently N or CR₆; each R₆ is independently hydrogen, cyano, halogen, OR₇, NR₈R₉, amino, hydroxyl, or optionally substituted alkyl, alkyl-aryl or alkyl-heterocycle; each R₇ is independently hydrogen or optionally substituted alkyl, alkyl-aryl or alkyl-heterocycle; each R₈ is independently hydrogen or optionally substituted alkyl, alkyl-aryl or alkyl-heterocycle; each R₉ is independently hydrogen or optionally substituted alkyl, alkyl-aryl or alkyl-heterocycle; and m is 1-4. Certain such compounds are of the formula:

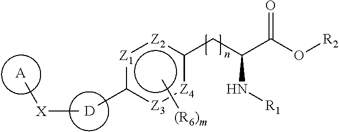

Others are of the formula:

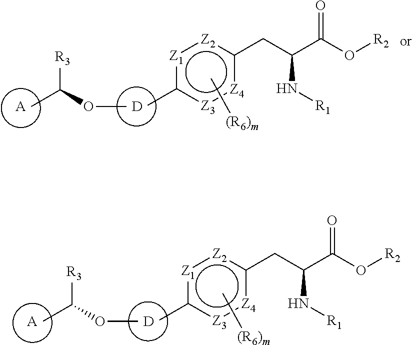

wherein, for example, R₃ is trifluoromethyl. Others are of the formula:

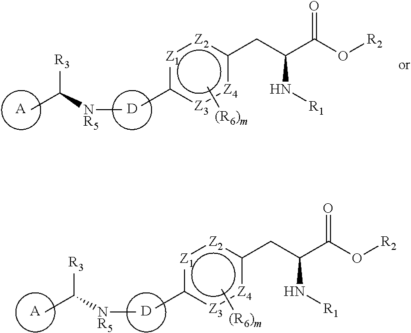

wherein, for example, R₃ is hydrogen.

Referring to the various formulae above, some compounds are such that all of Z₁, Z₂, Z₃, and Z₄ are N. In others, only three of Z₁, Z₂, Z₃, and Z₄ are N. In others, only two of Z₁, Z₂, Z₃, and Z₄ are N. In others, only one of Z₁, Z₂, Z₃, and Z₄ is N. In others, none of Z₁, Z₂, Z₃, and Z₄ are N.

Some compounds are of the formula:

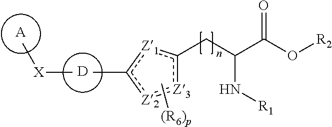

wherein: each of Z′₁, Z′₂, and Z′₃ is independently N, NH, S, O or CR₆; each R₆ is independently amino, cyano, halogen, hydrogen, OR₇, SR₇, NR₈R₉, or optionally substituted alkyl, alkyl-aryl or alkyl-heterocycle; each R₇ is independently hydrogen or optionally substituted alkyl, alkyl-aryl or alkyl-heterocycle; each R₈ is independently hydrogen or optionally substituted alkyl, alkyl-aryl or alkyl-heterocycle; each R₉ is independently hydrogen or optionally substituted alkyl, alkyl-aryl or alkyl-heterocycle; and p is 1-3. Certain such compounds are of the formula:

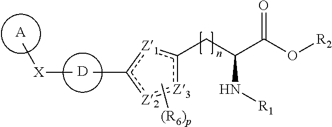

Others are of the formula:

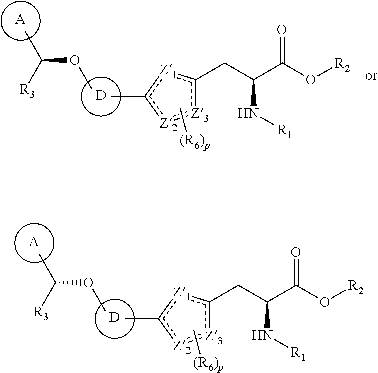

wherein, for example, R₃ is trifluoromethyl. Others are of the formula:

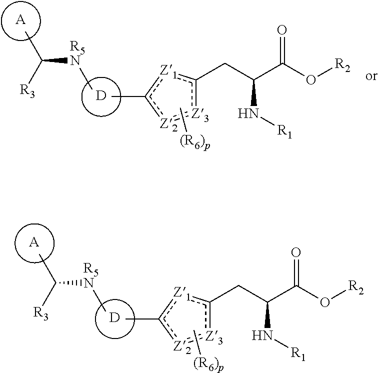

wherein, for example, R₃ is hydrogen.

Referring to the various formulae above, some compounds are such that all of Z′₁, Z′₂, and Z′₃ are N or NH. In others, only two of Z′₁, Z′₂, and Z′₃ are N or NH. In others, only one of Z′₁, Z′₂, and Z′₃ is N or NH. In others, none of Z′₁, Z′₂, and Z′₃ are N or NH.

Some compounds are encompassed by the formula:

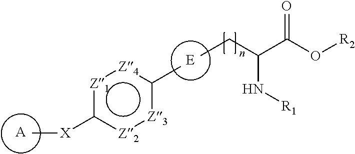

wherein: each of Z″₁, Z″₂, Z″₃, and Z″₄ is independently N or CR₁₀; each R₁₀ is independently amino, cyano, halogen, hydrogen, OR₁₁, SR₁₁, NR₁₂R₁₃, or optionally substituted alkyl, alkyl-aryl or alkyl-heterocycle; each R₁₁ is independently hydrogen or optionally substituted alkyl, alkyl-aryl or alkyl-heterocycle; each R₁₂ is independently hydrogen or optionally substituted alkyl, alkyl-aryl or alkyl-heterocycle; and each R₁₃ is independently hydrogen or optionally substituted alkyl, alkyl-aryl or alkyl-heterocycle. Certain such compounds are of the formula:

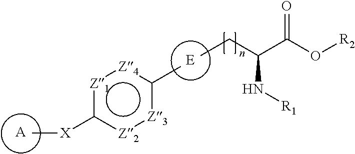

Others are of the formula:

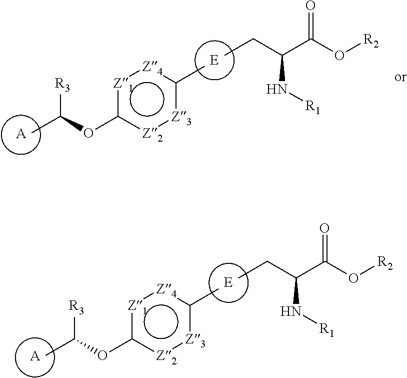

wherein, for example, R₃ is trifluoromethyl. Others are of the formula:

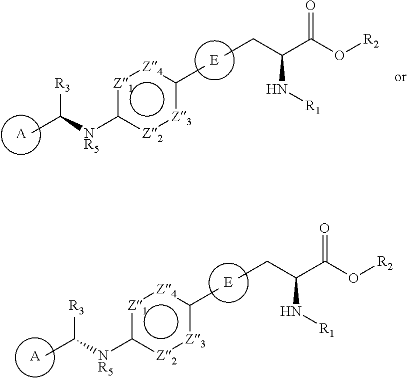

wherein, for example, R₃ is hydrogen.

Referring to the various formulae above, some compounds are such that all of Z″₁, Z″₂, Z″₃, and Z″₄ are N. In others, only three of Z″₁, Z″₂, Z″₃, and Z″₄ are N. In others, only two of Z″₁, Z″₂, Z″₃, and Z″₄ are N. In others, only one of Z″₁, Z″₂, Z″₃, and Z″₄ is N. In others, none of Z″₁, Z″₂, Z″₃, and Z″₄ are N.

Some compounds are of the formula:

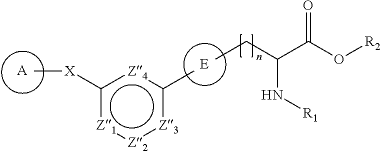

wherein: each of Z″₁, Z″₂, Z″₃, and Z″₄ is independently N or CR₁₀; each R₁₀ is independently amino, cyano, halogen, hydrogen, OR₁₁, SR₁₁, NR₁₂R₁₃, or optionally substituted alkyl, alkyl-aryl or alkyl-heterocycle; each R₁₁ is independently hydrogen or optionally substituted alkyl, alkyl-aryl or alkyl-heterocycle; each R₁₂ is independently hydrogen or optionally substituted alkyl, alkyl-aryl or alkyl-heterocycle; and each R₁₃ is independently hydrogen or optionally substituted alkyl, alkyl-aryl or alkyl-heterocycle. Certain such compounds are of the formula:

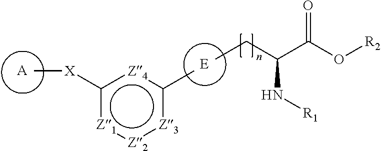

Others are of the formula:

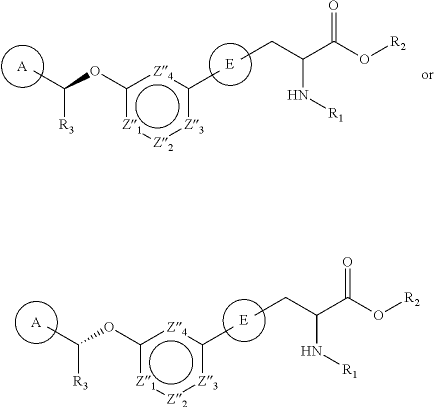

wherein, for example, R₃ is trifluoromethyl. Others are of the formula:

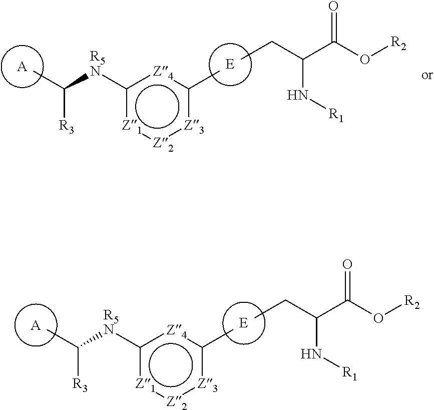

wherein, for example, R₃ is hydrogen.

Referring to the various formulae above, some compounds are such that all of Z″₁, Z″₂, Z″₃, and Z″₄ are N. In others, only three of Z″₁, Z″₂, Z″₃, and Z″₄ are N. In others, only two of Z″₁, Z″₂, Z″₃, and Z″₄ are N. In others, only one of Z″₁, Z″₂, Z″₃, and Z″₄ is N. In others, none of Z″₁, Z″₂, Z″₃, and Z″₄ are N.

Some are of the formula:

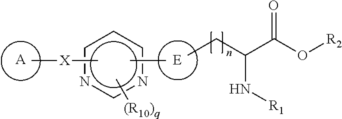

the substituents of which are defined herein.

Others are of the formula:

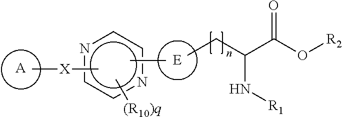

the substituents of which are defined herein.

Others are of the formula:

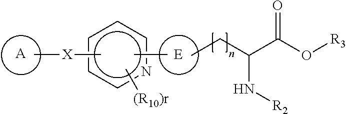

the substituents of which are defined herein.

Others are of the formula:

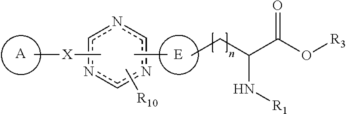

the substituents of which are defined herein.

Referring to the various formulae disclosed herein, particular compounds include those wherein both A and E are optionally substituted phenyl and, for example, X is —O—, —C(R₃R₄)O—, or —OC(R₃R₄)— and, for example, R₃ is hydrogen and R₄ is trifluoromethyl and, for example, n is 1.

Particular compounds of the invention are of formula III:

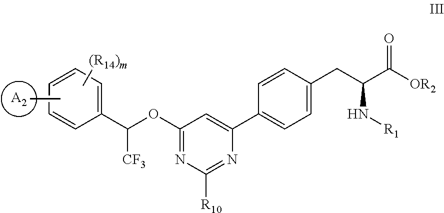

wherein: A₂ is optionally substituted heterocycle; R₁ is hydrogen, C(O)R_{A}, C(O)OR_{A}, or optionally substituted alkyl, alkyl-aryl, alkyl-heterocycle, aryl, or heterocycle; R₂ is hydrogen or optionally substituted alkyl, alkyl-aryl, alkyl-heterocycle, aryl, or heterocycle; R₁₀ is halogen, hydrogen, C(O)R_{A}, OR_{A}, NR_{B}R_{C}, S(O₂)R_{A}, or optionally substituted alkyl, alkyl-aryl or alkyl-heterocycle; each R₁₄ is independently halogen, hydrogen, C(O)R_{A}, OR_{A}, NR_{B}R_{C}, S(O₂)R_{A}, or optionally substituted alkyl, alkyl-aryl or alkyl-heterocycle; R_{A} is hydrogen or optionally substituted alkyl, alkyl-aryl or alkyl-heterocycle; R_{B} is hydrogen or optionally substituted alkyl, alkyl-aryl or alkyl-heterocycle; R_{C} is hydrogen or optionally substituted alkyl, alkyl-aryl or alkyl-heterocycle; and m is 1-4.

Some compounds are of the formula:

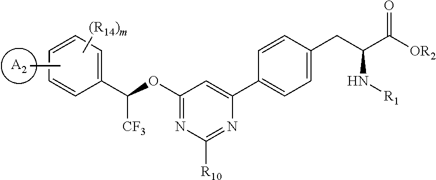

Some are of the formula:

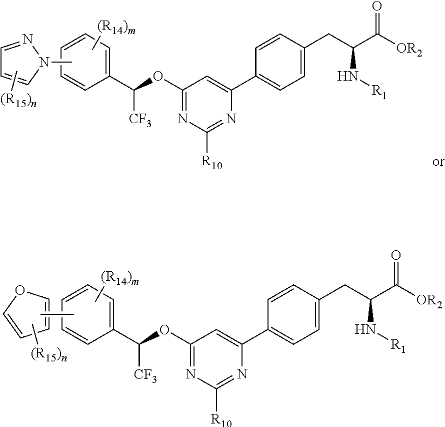

wherein: each R₁₅ is independently halogen, hydrogen, C(O)R_{A}, OR_{A}, NR_{B}R_{C}, S(O₂)R_{A}, or optionally substituted alkyl, alkyl-aryl or alkyl-heterocycle; and n is 1-3.

Some compounds are of the formula:

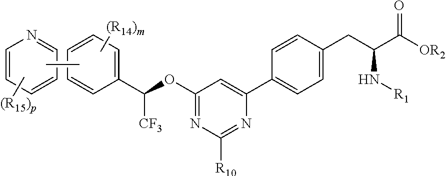

wherein: each R₁₅ is independently halogen, hydrogen, C(O)R_{A}, OR_{A}, NR_{B}R_{C}, S(O₂)R_{A}, or optionally substituted alkyl, alkyl-aryl or alkyl-heterocycle; and p is 1-4.

Others are of the formula:

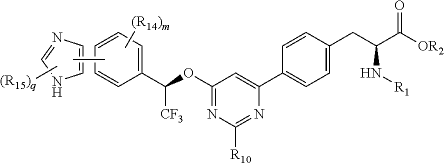

wherein: each R₁₅ is independently halogen, hydrogen, C(O)R_{A}, OR_{A}, NR_{B}R_{C}, S(O₂)R_{A}, or optionally substituted alkyl, alkyl-aryl or alkyl-heterocycle; and q is 1-2.

Some compounds are of the formula:

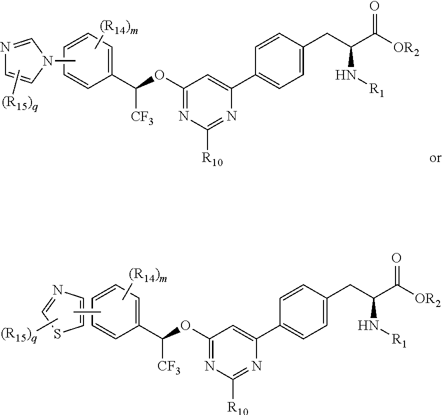

wherein: each R₁₅ is independently halogen, hydrogen, C(O)R_{A}, OR_{A}, NR_{B}R_{C}, S(O₂)R_{A}, or optionally substituted alkyl, alkyl-aryl or alkyl-heterocycle; and q is 1-2.

In particular compounds of formula III, A₂ is aromatic. In others, A₂ is not aromatic. In some, A₂ is optionally substituted with one or more of halogen or lower alkyl. In some, R₁₄ is hydrogen or halogen. In some, m is 1. In some, R₁₀ is hydrogen or amino. In some, R₁ is hydrogen or lower alkyl. In others, R₁ is C(O)OR_{A} and R_{A} is alkyl. In some, R₂ is hydrogen or lower alkyl. In some, R₁₅ is hydrogen or lower alkyl (e.g., methyl). In some, n is 1. In some, p is 1. In some, q is 1.

This invention encompasses stereomerically pure compounds and stereomerically enriched compositions of them. Stereoisomers may be asymmetrically synthesized or resolved using standard techniques such as chiral columns, chiral resolving agents, or enzymatic resolution. See, e.g., Jacques, J., et al., Enantiomers, Racemates and Resolutions (Wiley Interscience, New York, 1981); Wilen, S. H., et al., Tetrahedron 33:2725 (1977); Eliel, E. L., Stereochemistry of Carbon Compounds (McGraw Hill, N.Y., 1962); and Wilen, S. H., Tables of Resolving Agents and Optical Resolutions, p. 268 (E.L. Eliel, Ed., Univ. of Notre Dame Press, Notre Dame, Ind., 1972).

Particular compounds of the invention are potent TPH1 inhibitors. Specific compounds have a TPH1_IC₅₀ of less than about 10, 5, 2.5, 1, 0.75, 0.5, 0.4, 0.3, 0.2, 0.1, or 0.05 μM.

Particular compounds are selective TPH1 inhibitors. Specific compounds have a TPH1_IC₅₀ that is about 10, 25, 50, 100, 250, 500, or 1000 times less than their TPH2_IC₅₀.

Specific THP1 inhibitors include:
(S)-2-amino-3-(4-(5-(2-fluoro-4,5-dimethoxybenzylamino)pyrazin-2-yl)phenyl)propanoic acid;
(S)-2-amino-3-(4-(2-amino-6-(4-(2-methoxyphenyl)piperidin-1-yl)pyrimidin-4-yl)phenyl)propanoic acid;
(S)-2-amino-3-(4-(6-(3-(cyclopentyloxy)-4-methoxybenzylamino)-2-(dimethylamino)pyrimidin-4-yl)phenyl)propanoic acid;
(S)-2-amino-3-(4-(5-(3,4-dimethylbenzylamino)pyrazin-2-yl)phenyl)propanoic acid;
(S)-2-amino-3-(4-(5-(biphenyl-2-ylmethylamino)pyrazin-2-yl)phenyl)propanoic acid;
(S)-ethyl 2-amino-3-(4-(2-amino-6-(4-(trifluoromethyl)benzylamino)pyrimidin-4-yl)phenyl)propanoate;
(S)-2-amino-3-(4-(5-(cyclopentylmethylamino)pyrazin-2-yl)phenyl)propanoic acid;
(2S)-2-amino-3-(4-(2-amino-6-(3-(2-(trifluoromethyl)phenyl)pyrrolidin-1-yl)pyrimidin-4-yl)phenyl)propanoic acid;
(2S)-2-amino-3-(4-(2-amino-6-(1,2,3,4-tetrahydronaphthalen-1-ylamino)pyrimidin-4-yl)phenyl)propanoic acid;
(S)-2-amino-3-(4-(2-amino-6-((R)-1-(naphthalen-2-yl)ethoxy)pyrimidin-4-yl)phenyl)propanoic acid;
(2S)-2-amino-3-(4-(2-amino-6-(1,2-diphenylethylamino)pyrimidin-4-yl)phenyl)propanoic acid;
(S)-2-amino-3-(4-(2-amino-6-((R)-1-(4-(benzo[b]thiophen-3-yl)phenyl)ethylamino)pyrimidin-4-yl)phenyl)propanoic acid;
(S)-2-amino-3-(4-(4-amino-6-((R)-1-(4′-methoxybiphenyl-4-yl)ethylamino)-1,3,5-triazin-2-yl)phenyl)propanoic acid;
2-amino-3-(1-(4-amino-6-((R)-1-(naphthalen-2-yl)ethylamino)-1,3,5-triazin-2-yl)piperidin-4-yl)propanoic acid;
(2S)-2-amino-3-(4-(4-amino-6-(1-(4-fluoronaphthalen-1-yl)ethylamino)-1,3,5-triazin-2-yl)phenyl)propanoic acid;
(S)-2-amino-3-(4-(4-amino-6-((3′-fluorobiphenyl-4-yl)methylamino)-1,3,5-triazin-2-yl)phenyl)propanoic acid;
2-amino-3-(4-(4-amino-6-((R)-1-(naphthalen-2-yl)ethylamino)-1,3,5-triazin-2-yl)-2-fluorophenyl)propanoic acid;
(S)-2-amino-3-(4-(2-amino-6-((R)-2,2,2-trifluoro-1-(3′-methoxybiphenyl-4-yl)ethoxy)pyrimidin-4-yl)phenyl)propanoic acid;
(2S)-2-amino-3-(4-(4-amino-6-(2,2,2-trifluoro-1-(3′-fluorobiphenyl-2-yl)ethoxy)-1,3,5-triazin-2-yl)phenyl)propanoic acid;
(2S)-2-amino-3-(4-(4-amino-6-(1-(4-tert-butylphenyl)ethylamino)-1,3,5-triazin-2-yl)phenyl)propanoic acid;
(2S)-2-amino-3-(4-(2-amino-6-(2,2,2-trifluoro-1-(3′-fluorobiphenyl-4-yl)ethoxy)pyrimidin-4-yl)phenyl)propanoic acid;
(2S)-2-amino-3-(4-(4-amino-6-(6,7-dihydroxy-1-methyl-3,4-dihydroisoquinolin-2(1H)-yl)-1,3,5-triazin-2-yl)phenyl)propanoic acid;
(2S)-2-amino-3-(4-(4-amino-6-(2,2,2-trifluoro-1-(3′-methylbiphenyl-4-yl)ethoxy)-1,3,5-triazin-2-yl)phenyl)propanoic acid;
(S)-2-amino-3-(4-(4-amino-6-((R)-1-(naphthalen-2-yl)ethylamino)pyrimidin-2-yl)phenyl)propanoic acid;
(S)-2-amino-3-(4-(2-amino-6-(benzylthio)pyrimidin-4-yl)phenyl)propanoic acid;
(2S)-2-amino-3-(4-(2-amino-6-(2,2,2-trifluoro-1-(4′-fluorobiphenyl-4-yl)ethoxy)pyrimidin-4-yl)phenyl)propanoic acid;
(2S)-2-amino-3-(4-(6-(3-(4-chlorophenoxy)piperidin-1-yl)pyrimidin-4-yl)phenyl)propanoic acid;
(S)-3-(4-(4-amino-6-((R)-1-(naphthalen-2-yl)ethylamino)-1,3,5-triazin-2-yl)phenyl)-2-(2-aminoacetamido)propanoic acid;
(S)-2-amino-3-(4-(6-((R)-1-(naphthalen-2-yl)ethylamino)-2-(trifluoromethyl)pyrimidin-4-yl)phenyl)propanoic acid;
(S)-2-amino-3-(4-(2-amino-6-(4-(3-chlorophenyl)piperazin-1-yl)pyrimidin-4-yl)phenyl)propanoic acid;
(S)-2-amino-3-(4-(2-amino-6-((R)-2,2,2-trifluoro-1-phenylethoxy)pyrimidin-4-yl)phenyl)propanoic acid;
(2S)-2-amino-3-(4-(2-amino-6-(1,4-diphenylbutylamino)pyrimidin-4-yl)phenyl)propanoic acid;
(2S)-2-amino-3-(4-(6-(1-(3′-chlorobiphenyl-2-yl)-2,2,2-trifluoroethoxy)pyrimidin-4-yl)phenyl)propanoic acid;
(2S)-2-amino-3-(4-(4-amino-6-(1-(biphenyl-4-yl)-2,2,2-trifluoroethoxy)-1,3,5-triazin-2-yl)phenyl)propanoic acid;
(2S)-2-amino-3-(4-(2-amino-6-(2,2,3,3,3-pentafluoro-1-(3-fluoro-4-methylphenyl)propoxy)pyrimidin-4-yl)phenyl)propanoic acid;
(S)-ethyl 2-amino-3-(4-(2-amino-6-((R)-2,2,2-trifluoro-1-(3′-methoxybiphenyl-4-yl)ethoxy)pyrimidin-4-yl)phenyl)propanoate;
(S)-2-amino-3-(4-(2-amino-6-((S)-2,2,2-trifluoro-1-(3′-methoxybiphenyl-4-yl)ethoxy)pyrimidin-4-yl)phenyl)propanoic acid;
(2S)-2-amino-3-(4-(2-amino-6-(2,2,2-trifluoro-1-(3-fluoro-3′-methoxybiphenyl-4-yl)ethoxy)pyrimidin-4-yl)phenyl)propanoic acid;
(2S)-2-amino-3-(4-(2-amino-6-(1-(3′-(dimethylamino)biphenyl-2-yl)-2,2,2-trifluoroethoxy)pyrimidin-4-yl)phenyl)propanoic acid;
(2S)-2-amino-3-(4-(2-amino-6-(2,2,2-trifluoro-1-(3′-methoxy-5-methylbiphenyl-2-yl)ethoxy)pyrimidin-4-yl)phenyl)propanoic acid;
(2S)-2-amino-3-(4-(2-amino-6-(2,2,2-trifluoro-1-(4′-methoxy-5-methylbiphenyl-2-yl)ethoxy)pyrimidin-4-yl)phenyl)propanoic acid;
(2S)-2-amino-3-(4-(2-amino-6-(2,2,2-trifluoro-1-(3′-methoxy-3-(methylsulfonyl)biphenyl-4-yl)ethoxy)pyrimidin-4-yl)phenyl)propanoic acid;
(2S)-2-amino-3-(4-(2-amino-6-(1-(2-(cyclopropylmethoxy)-4-fluorophenyl)-2,2,2-trifluoroethoxy)pyrimidin-4-yl)phenyl)propanoic acid;
(2S)-2-amino-3-(4-(6-(1-(2-(cyclopropylmethoxy)-4-fluorophenyl)-2,2,2-trifluoroethoxy)pyrimidin-4-yl)phenyl)propanoic acid;
(2S)-2-amino-3-(4-(2-amino-6-(2,2,2-trifluoro-1-(2-(isopentyloxy)phenyl)ethoxy)pyrimidin-4-yl)phenyl)propanoic acid;
(2S)-2-amino-3-(4-(5-(2,2,2-trifluoro-1-(3′-fluorobiphenyl-4-yl)ethoxy)pyrazin-2-yl)phenyl)propanoic acid;
(2S)-2-amino-3-(4-(2-amino-6-(2,2,2-trifluoro-1-(4′-methoxybiphenyl-2-yl)ethoxy)pyrimidin-4-yl)phenyl)propanoic acid;
(2S)-2-amino-3-(4-(2-amino-6-(1-(3′-carbamoylbiphenyl-2-yl)-2,2,2-trifluoroethoxy)pyrimidin-4-yl)phenyl)propanoic acid;
(2S)-2-amino-3-(4-(2-amino-6-(1-(4′-carbamoylbiphenyl-2-yl)-2,2,2-trifluoroethoxy)pyrimidin-4-yl)phenyl)propanoic acid;
(2S)-2-amino-3-(4-(2-amino-6-(2,2,2-trifluoro-1-(4-(2-methoxyphenoxy)phenyl)ethoxy)pyrimidin-4-yl)phenyl)propanoic acid;
(2S)-2-amino-3-(4-(6-(2,2,2-trifluoro-1-(4-(2-methoxyphenoxy)phenyl)ethoxy)pyrimidin-4-yl)phenyl)propanoic acid;
(2S)-2-amino-3-(4-(6-(2,2,2-trifluoro-1-(2-(isopentyloxy)phenyl)ethoxy)pyrimidin-4-yl)phenyl)propanoic acid;
(2S)-3-(4-(6-(1-(3′-acetamidobiphenyl-2-yl)-2,2,2-trifluoroethoxy)-2-aminopyrimidin-4-yl)phenyl)-2-aminopropanoic acid;
(2S)-3-(4-(6-(1-(4′-acetamidobiphenyl-2-yl)-2,2,2-trifluoroethoxy)-2-aminopyrimidin-4-yl)phenyl)-2-aminopropanoic acid;
(2S)-2-amino-3-(4-(2-amino-6-(1-(4-cyanophenyl)-2,2,2-trifluoroethoxy)pyrimidin-4-yl)phenyl)propanoic acid;
(S)-ethyl 2-amino-3-(4-(2-amino-6-((R)-2,2,2-trifluoro-1-p-tolylethoxy)pyrimidin-4-yl)phenyl)propanoate;
(2S)-2-amino-3-(4-(2-amino-6-(2,2,2-trifluoro-1-(1-methoxybicyclo[2.2.2]oct-5-en-2-yl)ethoxy)pyrimidin-4-yl)phenyl)propanoic acid;
(2S)-2-amino-3-(4-(2-amino-6-(1-(4-(cyclopentyloxy)phenyl)-2,2,2-trifluoroethoxy)pyrimidin-4-yl)phenyl)propanoic acid;
(2S)-2-amino-3-(4-(6-(1-(4-(cyclopentyloxy)phenyl)-2,2,2-trifluoroethoxy)pyrimidin-4-yl)phenyl)propanoic acid;
(2S)-2-amino-3-(4-(2-amino-6-(2,2,2-trifluoro-1-(4-(3-methoxyphenoxy)phenyl)ethoxy)pyrimidin-4-yl)phenyl)propanoic acid;
(2S)-2-amino-3-(4-(2-amino-6-(1-(4,5-dimethoxybiphenyl-2-yl)-2,2,2-trifluoroethoxy)pyrimidin-4-yl)phenyl)propanoic acid;
(2S)-2-amino-3-(4-(2-amino-6-(1-(4,5-dimethoxy-3′-methylbiphenyl-2-yl)-2,2,2-trifluoroethoxy)pyrimidin-4-yl)phenyl)propanoic acid;
(2S)-2-amino-3-(4-(5-(2,2,2-trifluoro-1-(2′-methylbiphenyl-2-yl)ethoxy)pyrazin-2-yl)phenyl)propanoic acid;
(2S)-2-amino-3-(4-(6-(2,2,2-trifluoro-1-(4-(3-methoxyphenoxy)phenyl)ethoxy)pyrimidin-4-yl)phenyl)propanoic acid;
(2S)-2-amino-3-(4-(2-amino-6-(1-(2-(3,5-difluorophenoxy)phenyl)-2,2,2-trifluoroethoxy)pyrimidin-4-yl)phenyl)propanoic acid;
(2S)-2-amino-3-(4-(2-amino-6-(2,2,2-trifluoro-1-(4-(4-methoxyphenoxy)phenyl)ethoxy)pyrimidin-4-yl)phenyl)propanoic acid;
(2S)-2-amino-3-(4-(2-amino-6-(1-(4′-((S)-2-amino-2-carboxyethyl)biphenyl-2-yl)-2,2,2-trifluoroethoxy)pyrimidin-4-yl)phenyl)propanoic acid;
(2S)-2-amino-3-(4-(2-amino-6-(1-(2-bromophenyl)-2,2,2-trifluoroethoxy)pyrimidin-4-yl)phenyl)propanoic acid;
(2S)-2-amino-3-(4-(5-(2,2,2-trifluoro-1-(3′-methylbiphenyl-2-yl)ethoxy)pyrazin-2-yl)phenyl)propanoic acid;
(2S)-2-amino-3-(4-(2-amino-6-(2,2,2-trifluoro-1-(4-methoxybiphenyl-2-yl)ethoxy)pyrimidin-4-yl)phenyl)propanoic acid;
(2S)-2-amino-3-(4-(5-(2,2,2-trifluoro-1-(2-(4-methylthiophen-3-yl)phenyl)ethoxy)pyrazin-2-yl)phenyl)propanoic acid;
(2S)-2-amino-3-(4-(2-amino-6-(2,2,2-trifluoro-1-(4-methoxy-3′-methylbiphenyl-2-yl)ethoxy)pyrimidin-4-yl)phenyl)propanoic acid;
(2S)-2-amino-3-(4-(2-amino-6-(2,2,2-trifluoro-1-(3′-(hydroxymethyl)biphenyl-2-yl)ethoxy)pyrimidin-4-yl)phenyl)propanoic acid;
(2S)-2-amino-3-(4-(2-amino-6-(1-(3′-cyanobiphenyl-2-yl)-2,2,2-trifluoroethoxy)pyrimidin-4-yl)phenyl)propanoic acid;
(2S)-2-amino-3-(4-(6-(1-(2-(3,5-difluorophenoxy)phenyl)-2,2,2-trifluoroethoxy)pyrimidin-4-yl)phenyl)propanoic acid;
(2S)-2-amino-3-(4-(6-(2,2,2-trifluoro-1-(4-(4-methoxyphenoxy)phenyl)ethoxy)pyrimidin-4-yl)phenyl)propanoic acid;
(2S)-2-amino-3-(4-(2-amino-6-(2,2,2-trifluoro-1-(2-(4-methylthiazol-2-yl)thiophen-3-yl)ethoxy)pyrimidin-4-yl)phenyl)propanoic acid;
(2S)-2-amino-3-(4-(2-amino-6-(2,2,2-trifluoro-1-(5-(4-methoxyphenyl)isoxazol-3-yl)ethoxy)pyrimidin-4-yl)phenyl)propanoic acid;
(2S)-2-amino-3-(4-(2-amino-6-(2,2,2-trifluoro-1-(1-phenyl-5-(trifluoromethyl)-1H-pyrazol-4-yl)ethoxy)pyrimidin-4-yl)phenyl)propanoic acid;
(2S)-2-amino-3-(4-(2-amino-6-(1-(2-(cyclohexyloxy)-4-methylphenyl)-2,2,2-trifluoroethoxy)pyrimidin-4-yl)phenyl)propanoic acid;
(2S)-2-amino-3-(4-(2-amino-6-(1-(2-(cyclopentyloxy)-4-methylphenyl)-2,2,2-trifluoroethoxy)pyrimidin-4-yl)phenyl)propanoic acid;
(2S)-2-amino-3-(4-(2-amino-6-(1-(benzo[d]thiazol-6-yl)-2,2,2-trifluoroethoxy)pyrimidin-4-yl)phenyl)propanoic acid;
(2S)-2-amino-3-(4-(2-amino-6-(2,2,2-trifluoro-1-(1-methyl-1H-imidazol-5-yl)ethoxy)pyrimidin-4-yl)phenyl)propanoic acid;
(2S)-2-amino-3-(4-(6-(1-(2-(cyclopentyloxy)-4-methylphenyl)-2,2,2-trifluoroethoxy)pyrimidin-4-yl)phenyl)propanoic acid;
(2S)-2-amino-3-(4-(6-(1-(2-(cyclohexyloxy)-4-methylphenyl)-2,2,2-trifluoroethoxy)pyrimidin-4-yl)phenyl)propanoic acid;
(2S)-2-amino-3-(4-(2-amino-6-(2,2,2-trifluoro-1-(pyridin-3-yl)ethoxy)pyrimidin-4-yl)phenyl)propanoic acid;
(2S)-2-amino-3-(4-(2-amino-6-(1-(1,3-dimethyl-1H-pyrazol-5-yl)-2,2,2-trifluoroethoxy)pyrimidin-4-yl)phenyl)propanoic acid;
(S)-2-amino-3-(4-(2-amino-6-(3-hydroxyphenyl)pyrimidin-4-yl)phenyl)propanoic acid;
(2S)-2-amino-3-(4-(2-amino-6-(2,2,2-trifluoro-1-(3′-hydroxybiphenyl-2-yl)ethoxy)pyrimidin-4-yl)phenyl)propanoic acid;
(S)-2-amino-3-(4-(2-amino-6-(3,5-difluorophenyl)pyrimidin-4-yl)phenyl)propanoic acid;
(2S)-2-amino-3-(4-(2-amino-6-(1-(3′,5′-difluorobiphenyl-2-yl)-2,2,2-trifluoroethoxy)pyrimidin-4-yl)phenyl)propanoic acid;
(2S)-2-amino-3-(4-(6-(2,2,2-trifluoro-1-(3′-fluorobiphenyl-3-yl)ethoxy)pyrazin-2-yl)phenyl)propanoic acid;
(2S)-2-amino-3-(4-(2-amino-6-(1-(5-ethoxy-2-methyl-2,3-dihydrobenzofuran-6-yl)-2,2,2-trifluoroethoxy)pyrimidin-4-yl)phenyl)propanoic acid;
(2S)-2-amino-3-(4-(2-amino-6-(1-(benzofuran-5-yl)-2,2,2-trifluoroethoxy)pyrimidin-4-yl)phenyl)propanoic acid;
(2S)-2-amino-3-(4-(2-amino-6-(2,2,2-trifluoro-1-(2-m-tolylfuran-3-yl)ethoxy)pyrimidin-4-yl)phenyl)propanoic acid;
(S)-ethyl 3-(4-(2-amino-6-((R)-2,2,2-trifluoro-1-(3′-methoxybiphenyl-4-yl)ethoxy)pyrimidin-4-yl)phenyl)-2-(2-aminoacetamido)propanoate;
(2S)-2-amino-3-(4-(6-(2,2,2-trifluoro-1-(2-(4-methylthiophen-3-yl)phenyl)ethoxy)pyrazin-2-yl)phenyl)propanoic acid;
(2S)-2-amino-3-(4-(2-amino-6-(2,2,2-trifluoro-1-(5-methyl-3-phenylisoxazol-4-yl)ethoxy)pyrimidin-4-yl)phenyl)propanoic acid;
(S)-2-amino-3-(4-(2-amino-6-(3-(methylthio)phenyl)pyrimidin-4-yl)phenyl)propanoic acid;
(2S)-2-amino-3-(4-(2-amino-6-(2,2,2-trifluoro-1-(3′-(methylthio)biphenyl-2-yl)ethoxy)pyrimidin-4-yl)phenyl)propanoic acid;
(2S)-2-amino-3-(4-(2-amino-6-(1-(3′-((dimethylamino)methyl)biphenyl-2-yl)-2,2,2-trifluoroethoxy)pyrimidin-4-yl)phenyl)propanoic acid;
(S)-2-amino-3-(4-(2-amino-6-(3-(trifluoromethoxy)phenyl)pyrimidin-4-yl)phenyl)propanoic acid;
(2S)-2-amino-3-(4-(2-amino-6-(2,2,2-trifluoro-1-(3′-(trifluoromethoxy)biphenyl-2-yl)ethoxy)pyrimidin-4-yl)phenyl)propanoic acid;
(S)-3-(4-(2-amino-6-((R)-2,2,2-trifluoro-1-(3′-methoxybiphenyl-4-yl)ethoxy)pyrimidin-4-yl)phenyl)-2-(2-aminoacetamido)propanoic acid;
(2S)-2-amino-3-(4-(2-amino-6-(2,2,2-trifluoro-1-(1-methyl-5-phenyl-1H-pyrazol-4-yl)ethoxy)pyrimidin-4-yl)phenyl)propanoic acid;
(2S)-2-amino-3-(4-(2-amino-6-(2,2,2-trifluoro-1-(4-(methylsulfonyl)phenyl)ethoxy)pyrimidin-4-yl)phenyl)propanoic acid;
(S)-2-amino-3-(4-(2-amino-6-((R)-1-(3′-(dimethylamino)biphenyl-2-yl)-2,2,2-trifluoroethoxy)pyrimidin-4-yl)phenyl)propanoic acid;
(2S)-2-amino-3-(4-(2-amino-6-(1-(2-chloro-4-(methylsulfonyl)phenyl)-2,2,2-trifluoroethoxy)pyrimidin-4-yl)phenyl)propanoic acid;
(2S)-2-amino-3-(4-(2-amino-6-(2,2,2-trifluoro-1-(3-(furan-2-yl)thiophen-2-yl)ethoxy)pyrimidin-4-yl)phenyl)propanoic acid;
(2S)-2-amino-3-(4-(2-amino-6-(1-(2-(cyclopentyloxy)-4-fluorophenyl)-2,2,2-trifluoroethoxy)pyrimidin-4-yl)phenyl)propanoic acid;
(2S)-2-amino-3-(4-(2-amino-6-(2,2,2-trifluoro-1-(2-(3-methoxyphenyl)cyclohex-1-enyl)ethoxy)pyrimidin-4-yl)phenyl)propanoic acid;
(2S)-2-amino-3-(4-(2-amino-6-(2,2,2-trifluoro-1-(pyrimidin-5-yl)ethoxy)pyrimidin-4-yl)phenyl)propanoic acid;
(2S)-2-amino-3-(4-(5-(2,2,2-trifluoro-1-(3′-methoxybiphenyl-3-yl)ethoxy)pyrazin-2-yl)phenyl)propanoic acid;
(S)-2-amino-3-(4-(2-amino-6-((S)-1-(3′-(dimethylamino)biphenyl-2-yl)-2,2,2-trifluoroethoxy)pyrimidin-4-yl)phenyl)propanoic acid;
(2S)-2-amino-3-(4-(2-amino-6-(2,2,2-trifluoro-1-(2-(furan-2-carboxamido)phenyl)ethoxy)pyrimidin-4-yl)phenyl)propanoic acid;
(2S)-2-amino-3-(4-(2-amino-6-(1-(4-chloro-2-(methylsulfonyl)phenyl)-2,2,2-trifluoroethoxy)pyrimidin-4-yl)phenyl)propanoic acid;
(S)-isopropyl 2-amino-3-(4-(2-amino-6-((R)-2,2,2-trifluoro-1-(3′-methoxybiphenyl-4-yl)ethoxy)pyrimidin-4-yl)phenyl)propanoate;
(2S)-2-amino-3-(4-(6-(1-(2-(cyclopentyloxy)-4-fluorophenyl)-2,2,2-trifluoroethoxy)pyrimidin-4-yl)phenyl)propanoic acid;
(2S)-2-amino-3-(4-(6-(1-(2-(cyclohexyloxy)-4-fluorophenyl)-2,2,2-trifluoroethoxy)pyrimidin-4-yl)phenyl)propanoic acid;
(2S)-2-amino-3-(4-(2-amino-6-(2,2,2-trifluoro-1-(1-(thiophen-2-yl)cyclohexyl)ethoxy)pyrimidin-4-yl)phenyl)propanoic acid;
(2S)-2-amino-3-(4-(2-(2,2,2-trifluoro-1-(3′-methoxybiphenyl-4-yl)ethoxy)thiazol-5-yl)phenyl)propanoic acid;
(2S)-2-amino-3-(4-(2-amino-6-(1-(2-(cyclohexyloxy)-4-fluorophenyl)-2,2,2-trifluoroethoxy)pyrimidin-4-yl)phenyl)propanoic acid;
(2S)-2-amino-3-(4-(2-amino-6-(2,2,2-trifluoro-1-(1-(4-methoxyphenyl)cyclohexyl)ethoxy)pyrimidin-4-yl)phenyl)propanoic acid;
(2S)-2-amino-3-(4-(6-(2,2,2-trifluoro-1-(4-fluoro-2-methylphenyl)ethoxy)pyrimidin-4-yl)phenyl)propanoic acid;
(2S)-2-amino-3-(4-(2-amino-6-(2,2,2-trifluoro-1-(4-fluoro-2-methylphenyl)ethoxy)pyrimidin-4-yl)phenyl)propanoic acid;
(2S)-2-amino-3-(4-(2-amino-6-(oxazol-2-yl(phenyl)methoxy)pyrimidin-4-yl)phenyl)propanoic acid;
(S)-2-amino-3-(4-(2-amino-6-(1-cyclohexyl-2,2,2-trifluoroethylideneaminooxy)pyrimidin-4-yl)phenyl)propanoic acid;
(2S)-2-amino-3-(4-(2-amino-6-(1-(2-(3-(dimethylamino)phenyl)furan-3-yl)-2,2,2-trifluoroethoxy)pyrimidin-4-yl)phenyl)propanoic acid;
(2S)-2-amino-3-(4-(2-amino-6-(2,2,2-trifluoro-1-(5-phenylthiophen-2-yl)ethoxy)pyrimidin-4-yl)phenyl)propanoic acid;
(S)-phenyl 2-amino-3-(4-(2-amino-6-((R)-2,2,2-trifluoro-1-(3′-methoxybiphenyl-4-yl)ethoxy)pyrimidin-4-yl)phenyl)propanoate;
(S)-2-amino-3-(4-(2-amino-6-((R)-1-(3′-((dimethylamino)methyl)biphenyl-4-yl)-2,2,2-trifluoroethoxy)pyrimidin-4-yl)phenyl)propanoic acid;
(S)-2-amino-3-(4-(1-(3-methoxybenzoyl)-1H-pyrazol-4-yl)phenyl)propanoic acid;
(2S)-2-amino-3-(4-(6-(2,2,2-trifluoro-1-(5-phenylfuran-2-yl)ethoxy)pyrimidin-4-yl)phenyl)propanoic acid;
(2S)-2-amino-3-(4-(2-amino-6-(1-(4-chloro-2-fluorophenyl)-2,2,2-trifluoroethoxy)pyrimidin-4-yl)phenyl)propanoic acid;
(S,E)-2-amino-3-(4-(2-amino-6-(4-(trifluoromethyl)styryl)pyrimidin-4-yl)phenyl)propanoic acid;
(2S)-2-amino-3-(4-(2-amino-6-(1-(3,4-dichlorophenyl)-2,2,2-trifluoroethoxy)pyrimidin-4-yl)phenyl)propanoic acid;
(2S)-2-amino-3-(4-(2-amino-6-(1-(4-chloro-3-fluorophenyl)-2,2,2-trifluoroethoxy)pyrimidin-4-yl)phenyl)propanoic acid;
(S)-2-amino-3-(4-(2-amino-6-((R)-1-(3′-(dimethylamino)biphenyl-4-yl)-2,2,2-trifluoroethoxy)pyrimidin-4-yl)phenyl)propanoic acid;
(2S)-2-amino-3-(4-(2-amino-6-(1-chloro-2,2,2-trifluoro-1-(4-methoxybiphenyl-2-yl)ethoxy)pyrimidin-4-yl)phenyl)propanoic acid;
(2S)-2-amino-3-(4-(6-(2,2,2-trifluoro-1-(5-phenylthiophen-2-yl)ethoxy)pyrimidin-4-yl)phenyl)propanoic acid;
(S)-2-amino-3-(4-(5-(4-phenoxyphenyl)-1H-1,2,3-triazol-1-yl)phenyl)propanoic acid;
(S,E)-2-amino-3-(4-(2-amino-6-(2-(biphenyl-4-yl)vinyl)pyrimidin-4-yl)phenyl)propanoic acid;
(S)-2-amino-3-(4-(4-amino-6-((R)-2,2,2-trifluoro-1-(3′-methoxybiphenyl-4-yl)ethoxy)pyrimidin-2-yl)phenyl)propanoic acid;
(S)-2-amino-3-(4-(4′-methoxybiphenyl-4-ylsulfonamido)phenyl)propanoic acid;
(2S)-2-amino-3-(4-(2-amino-6-(2,2,2-trifluoro-1-(6-(3-methoxyphenyl)pyridin-3-yl)ethoxy)pyrimidin-4-yl)phenyl)propanoic acid;
(2S)-2-amino-3-(4-(2-amino-6-(2,2,2-trifluoro-1-(6-(2-fluoro-3-methoxyphenyl)pyridin-3-yl)ethoxy)pyrimidin-4-yl)phenyl)propanoic acid;
2-amino-3-(5-(4′-methylbiphenyl-4-yl)-1H-indol-3-yl)propanoic acid;
2-amino-3-(5-m-tolyl-1H-indol-3-yl)propanoic acid;
(2S)-2-amino-3-(4-(2-(2-methoxyphenyl)furan-3-carboxamido)phenyl)propanoic acid;
2-amino-3-(5-(1-benzyl-1H-pyrazol-4-yl)-1H-indol-3-yl)propanoic acid;
(2S)-2-amino-3-(4-(2-amino-6-(2,2,2-trifluoro-1-(6-(thiophen-2-yl)pyridin-3-yl)ethoxy)pyrimidin-4-yl)phenyl)propanoic acid;
2-amino-3-(6-(1-benzyl-1H-pyrazol-4-yl)-1H-indol-3-yl)propanoic acid;
(S)-2-amino-3-(4-((2-(4-(trifluoromethyl)phenyl)thiazol-4-yl)methylamino)phenyl)propanoic acid;
(S)-2-amino-3-(4-((4′-methoxybiphenyl-4-ylsulfonamido)methyl)phenyl)propanoic acid;
(S)-2-amino-3-(4-(3-(2-methoxydibenzo[b,d]furan-3-yl)ureido)phenyl)propanoic acid;
(S)-2-amino-3-(4-(3-(2,2-diphenylethyl)ureido)phenyl)propanoic acid;
(S)-2-amino-3-(4-(phenylethynyl)phenyl)propanoic acid;
(S)-2-amino-3-(4-(2-amino-6-((5-(1-methyl-5-(trifluoromethyl)-1H-pyrazol-3-yl)thiophen-2-yl)methoxy)pyrimidin-4-yl)phenyl)propanoic acid;
(2S)-2-amino-3-(4-(2-amino-6-(1,1,1-trifluoro-3-((R)-2,2,3-trimethylcyclopent-3-enyl)propan-2-yloxy)pyrimidin-4-yl)phenyl)propanoic acid;
(2S)-2-amino-3-(4-(2-amino-6-(3-(2-hydroxyethylcarbamoyl)piperidin-1-yl)pyrimidin-4-yl)phenyl)propanoic acid;
(2S)-2-amino-3-(4-(2-amino-6-(3-(pyridin-2-yloxy)piperidin-1-yl)pyrimidin-4-yl)phenyl)propanoic acid;
(S)-2-amino-3-(4-(2-amino-6-(4-chloro-3-(piperidine-1-carbonyl)phenyl)pyrimidin-4-yl)phenyl)propanoic acid;
(2S)-2-amino-3-(4-(2-amino-6-(2,2,2-trifluoro-1-(4-(pyridin-3-yl)phenyl)ethoxy)pyrimidin-4-yl)phenyl)propanoic acid;
(2S)-2-amino-3-(4-(2-amino-6-(2,2,2-trifluoro-1-(2-(2-methylpyridin-4-yl)phenyl)ethoxy)pyrimidin-4-yl)phenyl)propanoic acid;
(2S)-2-amino-3-(4-(2-amino-6-(2,2,2-trifluoro-1-(2-(4-methylthiophen-3-yl)phenyl)ethoxy)pyrimidin-4-yl)phenyl)propanoic acid;
(2S)-3-(4-(6-(1-(2-(1H-pyrazol-1-yl)phenyl)-2,2,2-trifluoroethoxy)-2-aminopyrimidin-4-yl)phenyl)-2-aminopropanoic acid;
(2S)-2-amino-3-(4-(2-amino-6-(2,2,2-trifluoro-1-(4-(furan-2-yl)phenyl)ethoxy)pyrimidin-4-yl)phenyl)propanoic acid;
(2S)-2-amino-3-(4-(6-(2,2,2-trifluoro-1-(2-(pyridin-3-yloxy)phenyl)ethoxy)pyrimidin-4-yl)phenyl)propanoic acid;
(2S)-3-(4-(6-(1-(2-(1H-1,2,4-triazol-1-yl)phenyl)-2,2,2-trifluoroethoxy)-2-aminopyrimidin-4-yl)phenyl)-2-aminopropanoic acid;
(2S)-2-amino-3-(4-(2-amino-6-(2,2,2-trifluoro-1-(2-(furan-3-yl)phenyl)ethoxy)pyrimidin-4-yl)phenyl)propanoic acid;
(2S)-2-amino-3-(4-(2-amino-6-(2,2,2-trifluoro-1-(4-(furan-2-yl)-3-methoxyphenyl)ethoxy)pyrimidin-4-yl)phenyl)propanoic acid;
(2S)-2-amino-3-(4-(5-(2,2,2-trifluoro-1-(2-(furan-2-yl)phenyl)ethoxy)pyrazin-2-yl)phenyl)propanoic acid;
(2S)-3-(4-(5-(1-(2-(1H-pyrazol-1-yl)phenyl)-2,2,2-trifluoroethoxy)pyrazin-2-yl)phenyl)-2-aminopropanoic acid;
(2S)-2-amino-3-(4-(2-amino-6-(1-(4,5-dimethoxy-2-(1H-pyrazol-1-yl)phenyl)-2,2,2-trifluoroethoxy)pyrimidin-4-yl)phenyl)propanoic acid;
(2S)-2-amino-3-(4-(2-amino-6-(2,2,2-trifluoro-1-(2-(2-methyl-1H-imidazol-1-yl)phenyl)ethoxy)pyrimidin-4-yl)phenyl)propanoic acid;
(2S)-2-amino-3-(4-(2-amino-6-(2,2,2-trifluoro-1-(2-(5-methylthiophen-2-yl)phenyl)ethoxy)pyrimidin-4-yl)phenyl)propanoic acid;
(2S)-2-amino-3-(4-(2-amino-6-(1-(2-(5-(dimethylcarbamoyl)furan-2-yl)phenyl)-2,2,2-trifluoroethoxy)pyrimidin-4-yl)phenyl)propanoic acid;
(2S)-2-amino-3-(4-(2-amino-6-(2,2,2-trifluoro-1-(4-fluoro-2-(thiophen-2-yl)phenyl)ethoxy)pyrimidin-4-yl)phenyl)propanoic acid;
(2S)-2-amino-3-(4-(6-(2,2,2-trifluoro-1-(4-fluoro-2-(thiophen-2-yl)phenyl)ethoxy)pyrimidin-4-yl)phenyl)propanoic acid;
(2S)-2-amino-3-(4-(6-(2,2,2-trifluoro-1-(4-fluoro-2-(thiophen-3-yl)phenyl)ethoxy)pyrimidin-4-yl)phenyl)propanoic acid;
(2S)-2-amino-3-(4-(6-(2,2,2-trifluoro-1-(4-fluoro-2-(4-methylthiophen-2-yl)phenyl)ethoxy)pyrimidin-4-yl)phenyl)propanoic acid;
(S)-2-amino-3-(4-(2-amino-6-((R)-2,2,2-trifluoro-1-(4-(6-fluoropyridin-3-yl)phenyl)ethoxy)pyrimidin-4-yl)phenyl)propanoic acid;
(2S)-3-(4-(6-(1-(4-(1H-imidazol-1-yl)phenyl)-2,2,2-trifluoroethoxy)-2-aminopyrimidin-4-yl)phenyl)-2-aminopropanoic acid;
(2S)-2-amino-3-(4-(6-(2,2,2-trifluoro-1-(4-(thiophen-2-yl)phenyl)ethoxy)pyrimidin-4-yl)phenyl)propanoic acid;
(S)-2-amino-3-(4-(2-amino-6-((R)-2,2,2-trifluoro-1-(4-(pyrimidin-5-yl)phenyl)ethoxy)pyrimidin-4-yl)phenyl)propanoic acid;
(2S)-2-amino-3-(4-(6-(1-(2-(3,5-dimethylisoxazol-4-yl)-4-fluorophenyl)-2,2,2-trifluoroethoxy)pyrimidin-4-yl)phenyl)propanoic acid;
(S)-2-amino-3-(4-(2-amino-6-((R)-2,2,2-trifluoro-1-(4-(2-methylpyridin-4-yl)phenyl)ethoxy)pyrimidin-4-yl)phenyl)propanoic acid;
(2S)-3-(4-(6-(1-(4-(1H-1,2,4-triazol-1-yl)phenyl)-2,2,2-trifluoroethoxy)pyrimidin-4-yl)phenyl)-2-aminopropanoic acid;
(2S)-2-amino-3-(4-(2-amino-6-(2,2,2-trifluoro-1-(4-(piperidin-1-ylmethyl)phenyl)ethoxy)pyrimidin-4-yl)phenyl)propanoic acid;
(2S)-2-amino-3-(4-(2-amino-6-(2,2,2-trifluoro-1-(2-fluoro-4-(2-methylpyridin-4-yl)phenyl)ethoxy)pyrimidin-4-yl)phenyl)propanoic acid;
(2S)-2-amino-3-(4-(2-amino-6-(1-(4-(6-chloropyridazin-3-yl)phenyl)-2,2,2-trifluoroethoxy)pyrimidin-4-yl)phenyl)propanoic acid;
(2S)-2-amino-3-(4-(2-amino-6-(1-(4-(4-tert-butylthiazol-2-yl)phenyl)-2,2,2-trifluoroethoxy)pyrimidin-4-yl)phenyl)propanoic acid;
(2S)-2-amino-3-(4-(2-amino-6-(2,2,2-trifluoro-1-(3′-methoxy-3-(3-methyl-1H-pyrazol-1-yl)biphenyl-4-yl)ethoxy)pyrimidin-4-yl)phenyl)propanoic acid; and
(2S)-2-amino-3-(4-(2-amino-6-(1-(5-chloro-2-(3-methyl-1H-pyrazol-1-yl)phenyl)-2,2,2-trifluoroethoxy)pyrimidin-4-yl)phenyl)propanoic acid.

Particular THP1 are: (S)-2-amino-3-(4-(2-amino-6-((R)-2,2,2-trifluoro-1-(3′-methoxybiphenyl-4-yl)ethoxy)pyrimidin-4-yl)phenyl)propanoic acid; (S)-ethyl 2-amino-3-(4-(2-amino-6-((R)-1-(4-chloro-2-(3-methyl-1H-pyrazol-1-yl)phenyl)-2,2,2-trifluoroethoxy)pyrimidin-4-yl)phenyl)propanoate; and (S)-2-amino-3-(4-(2-amino-6-((R)-1-(4-chloro-2-(3-methyl-1H-pyrazol-1-yl)phenyl)-2,2,2-trifluoroethoxy)pyrimidin-4-yl)phenyl)propanoic acid, and pharmaceutically acceptable salts thereof.

When administered to mammals (e.g., mice, rats, dogs, monkeys or humans), certain compounds of the invention do not readily cross the blood/brain barrier (e.g., less than about 5, 2.5, 2, 1.5, 1, 0.5, or 0.01 percent of compound in the blood passes into the brain). The ability or inability of a compound to cross the blood/brain barrier can be determined by methods known in the art. See, e.g., Riant, P. et al., Journal of Neurochemistry 51:421-425 (1988); Kastin, A. J., Akerstrom, V., J. Pharmacol. Exp. Therapeutics 294:633-636 (2000); W. A. Banks, W. A., et al., J. Pharmacol. Exp. Therapeutics 302:1062-1069 (2002).

### 5.3. Methods of Use

This invention encompasses methods of treating, managing and/or preventing necrotizing enterocolitis, which comprise administering to a patient in need thereof a therapeutically or prophylactically effective amount of a TPH inhibitor. Particular patents are infants. Patients at risk of NEC, to whom a TPH inhibitor can be given to help prevent the disease, are premature infants and infants weighing less than 1.5 kg. Preferred TPH inhibitors are the compounds disclosed herein.

In particular methods, the TPH inhibitor is administered intravenously.

### 5.4. Pharmaceutical Compositions

This invention encompasses pharmaceutical compositions comprising one or more compounds of the invention. Certain pharmaceutical compositions are single unit dosage forms suitable for oral, mucosal (e.g., nasal, sublingual, vaginal, buccal, or rectal), parenteral (e.g., subcutaneous, intravenous, bolus injection, intramuscular, or intraarterial), or transdermal administration to a patient. Examples of dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The formulation should suit the mode of administration. For example, the oral administration of a compound susceptible to degradation in the stomach may be achieved using an enteric coating. Similarly, a formulation may contain ingredients that facilitate delivery of the active ingredient(s) to the site of action. For example, compounds may be administered in liposomal formulations in order to protect them from degradative enzymes, facilitate transport in circulatory system, and effect their delivery across cell membranes.

Similarly, poorly soluble compounds may be incorporated into liquid dosage forms (and dosage forms suitable for reconstitution) with the aid of solubilizing agents, emulsifiers and surfactants such as, but not limited to, cyclodextrins (e.g., α-cyclodextrin, β-cyclodextrin, Captisol®, and Encapsin™ (see, e.g., Davis and Brewster, Nat. Rev. Drug Disc. 3:1023-1034 (2004)), Labrasol®, Labrafil®, Labrafac®, cremafor, and non-aqueous solvents, such as, but not limited to, ethyl alcohol, isopropyl alcohol, ethyl carbonate, ethyl acetate, benzyl alcohol, benzyl benzoate, propylene glycol, 1,3-butylene glycol, dimethyl formamide, dimethyl sulfoxide (DMSO), biocompatible oils (e.g., cottonseed, groundnut, corn, germ, olive, castor, and sesame oils), glycerol, tetrahydrofurfuryl alcohol, polyethylene glycols, fatty acid esters of sorbitan, and mixtures thereof (e.g., DMSO:cornoil).

Poorly soluble compounds may also be incorporated into suspensions using other techniques known in the art. For example, nanoparticles of a compound may be suspended in a liquid to provide a nanosuspension (see, e.g., Rabinow, Nature Rev. Drug Disc. 3:785-796 (2004)). Nanoparticle forms of compounds described herein may be prepared by the methods described in U.S. Patent Publication Nos. 2004-0164194, 2004-0195413, 2004-0251332, 2005-0042177 A1, 2005-0031691 A₁, and U.S. Pat. Nos. 5,145,684, 5,510,118, 5,518,187, 5,534,270, 5,543,133, 5,662,883, 5,665,331, 5,718,388, 5,718,919, 5,834,025, 5,862,999, 6,431,478, 6,742,734, 6,745,962, the entireties of each of which are incorporated herein by reference. In one embodiment, the nanoparticle form comprises particles having an average particle size of less than about 2000 nm, less than about 1000 nm, or less than about 500 nm.

The composition, shape, and type of a dosage form will typically vary depending with use. For example, a dosage form used in the acute treatment of a disease may contain larger amounts of one or more of the active ingredients it comprises than a dosage form used in the chronic treatment of the same disease. Similarly, a parenteral dosage form may contain smaller amounts of one or more of the active ingredients it comprises than an oral dosage form used to treat the same disease. How to account for such differences will be apparent to those skilled in the art. See, e.g., Remington's Pharmaceutical Sciences, 18th ed., Mack Publishing, Easton Pa. (1990).

### 5.4.1. Oral Dosage Forms

Pharmaceutical compositions of the invention suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington's Pharmaceutical Sciences, 18th ed., Mack Publishing, Easton Pa. (1990).

Typical oral dosage forms are prepared by combining the active ingredient(s) in an intimate admixture with at least one excipient according to conventional pharmaceutical compounding techniques. Excipients can take a wide variety of forms depending on the form of preparation desired for administration.

Because of their ease of administration, tablets and capsules represent the most advantageous oral dosage unit forms. If desired, tablets can be coated by standard aqueous or non-aqueous techniques. Such dosage forms can be prepared by conventional methods of pharmacy. In general, pharmaceutical compositions and dosage forms are prepared by uniformly and intimately admixing the active ingredients with liquid carriers, finely divided solid carriers, or both, and then shaping the product into the desired presentation if necessary. Disintegrants may be incorporated in solid dosage forms to facility rapid dissolution. Lubricants may also be incorporated to facilitate the manufacture of dosage forms (e.g., tablets).

### 5.4.2. Parenteral Dosage Forms

Parenteral dosage forms can be administered to patients by various routes including subcutaneous, intravenous (including bolus injection), intramuscular, and intraarterial. Because their administration typically bypasses patients' natural defenses against contaminants, parenteral dosage forms are specifically sterile or capable of being sterilized prior to administration to a patient. Examples of parenteral dosage forms include solutions ready for injection, dry products ready to be dissolved or suspended in a pharmaceutically acceptable vehicle for injection, suspensions ready for injection, and emulsions.

Suitable vehicles that can be used to provide parenteral dosage forms of the invention are well known to those skilled in the art. Examples include: Water for Injection USP; aqueous vehicles such as Sodium Chloride Injection, Ringer's Injection, Dextrose Injection, Dextrose and Sodium Chloride Injection, and Lactated Ringer's Injection; water-miscible vehicles such as ethyl alcohol, polyethylene glycol, and polypropylene glycol; and non-aqueous vehicles such as corn oil, cottonseed oil, peanut oil, sesame oil, ethyl oleate, isopropyl myristate, and benzyl benzoate.

### 6. EXAMPLES

### 6.1. Treatment of Necrotizing Enterocolitis

One aspect of this experiment utilized knockout mice for the serotonin reuptake transporter (SERT) and for TPH1. Such knockout mice are well known, and can be obtained commercially and by methods known in the art. See, e.g., Chen, J. J., et al., J. Neuroscience 21(16):6348-6361 (2001); Liu, Q., et al., JPET 325(1):47-55 (2008).

Ten day-old breastfed SERT knockout mice, TPH1 knockout mice and wildtype littermate mice (C57BL/6) were subjected to a 5 day protocol of formula (gavage) feeding and hypoxia. This protocol reproducibly induces NEC in mice. Weights of all groups were monitored daily and cytokines were analyzed in the distal ileum at day 5. This experiment was then repeated using two groups of mice that received 100 mg/kg or 200 mg/kg of (2S)-ethyl 2-amino-3-(4-(2-amino-6-(2,2,2-trifluoro-1-(3′-fluoro-[1,1′-biphenyl]-4-yl)ethoxy)pyrimidin-4-yl)phenyl)propanoate (LP-920540), and a control group that received vehicle.

During the five days of the experiment, SERT knockout mice lost significantly more weight than their wildtype littermates (p<0.0001), while TPH1 knockout mice lost significantly less weight than their wildtype littermates (p<0.0006). The proinflammatory mediators, iNOS and IL18, were found to be significantly more upregulated in the SERT knockout mice than in their wildetype littermates, while the proinflammatory mediators, IL18 and IL6, were found to be significantly less upregulated in TPH1KO mice than in the wildtype littermates.

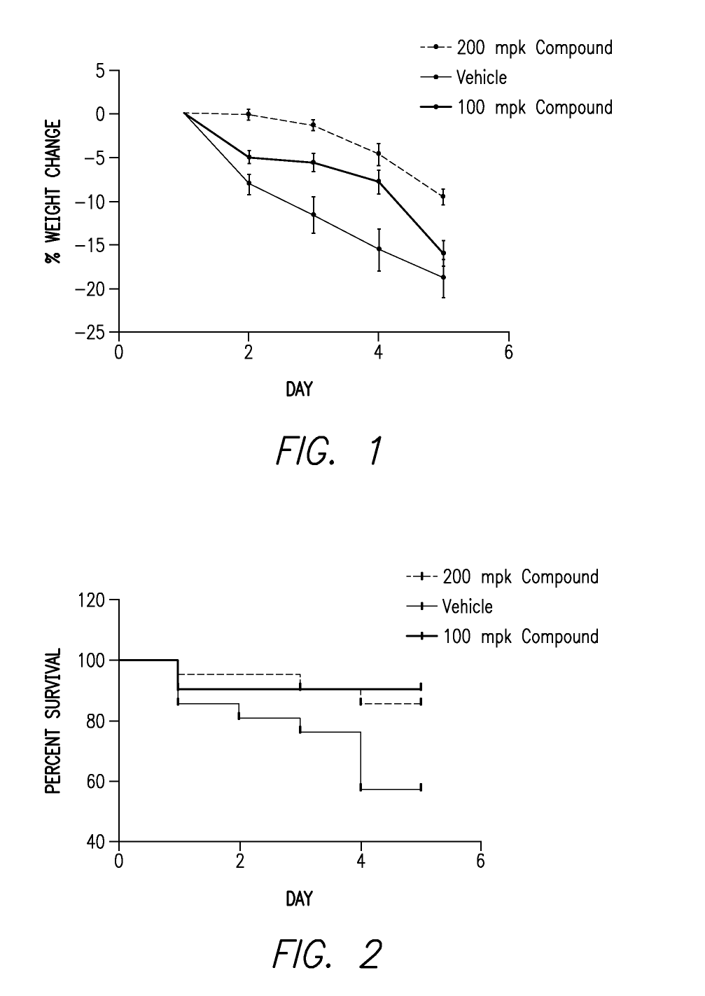

As shown in FIG. 1, mice that received either 100 or 200 mg/kg of LP-920540 lost significantly less weight than did vehicle-treated animals. As shown in FIG. 2, mortality (5-10%) was also better than that of vehicle treated animals (50%). Levels of transcripts encoding IL-18 and IL-6 were also significantly less upregulated in LP-920540 than in vehicle-treated mice.

All of the references (e.g., patents and patent applications) cited herein are incorporated herein in their entireties.

What is claimed is:

1. A compound of the formula: 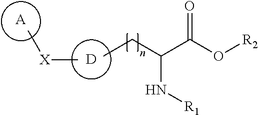
or a pharmaceutically acceptable salt thereof, for use in treating, managing or preventing necrotizing enterocolitis (NEC), wherein:
A is optionally substituted cycloalkyl, aryl, or heterocycle;
X is a bond, —O—, —S—, —C(O)—, —C(R₄)═, ═C(R₄)—, —C(R₃R₄)—, —C(R₄)═C(R₄)—, —C≡C—, —N(R₅)—, —N(R₅)C(O)N(R₅)—, —C(R₃R₄)N(R₅)—, —N(R₅)C(R₃R₄)—, —ONC(R₃)—, —C(R₃)NO—, —C(R₃R₄)O—, —OC(R₃R₄)—, —S(O₂)—, —S(O₂)N(R₅)—, —N(R₅)S(O₂)—, —C(R₃R₄)S(O₂)—, or —S(O₂)C(R₃R₄)—;
D is optionally substituted aryl or heterocycle;
R₁ is hydrogen or optionally substituted alkyl, alkyl-aryl, alkyl-heterocycle, aryl, or heterocycle;
R₂ is hydrogen or optionally substituted alkyl, alkyl-aryl, alkyl-heterocycle, aryl, or heterocycle;
R₃ is hydrogen, alkoxy, amino, cyano, halogen, hydroxyl, or optionally substituted alkyl;
R₄ is hydrogen, alkoxy, amino, cyano, halogen, hydroxyl, or optionally substituted alkyl or aryl;
each R₅ is independently hydrogen or optionally substituted alkyl or aryl; and
n is 0-3.

2. The use of claim 1, wherein the compound is of the formula:

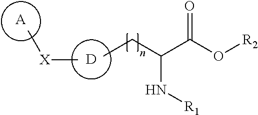

3. The use of claim 2, wherein the compound is of the formula: 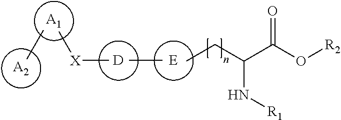
wherein: each of A₁ and A₂ is independently a monocyclic optionally substituted cycloalkyl, aryl, or heterocycle; and E is optionally substituted aryl or heterocycle.

4. The use of claim 3, wherein the compound is of the formula: 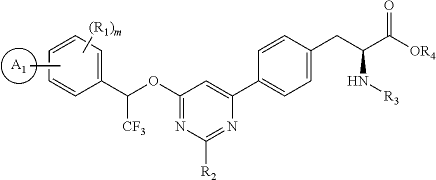
each R₁ is independently halogen, hydrogen, C(O)R_{A}, OR_{A}, NR_{B}R_{C}, S(O₂)R_{A}, or optionally substituted alkyl, alkyl-aryl or alkyl-heterocycle;
R₂ is independently halogen, hydrogen, C(O)R_{A}, OR_{A}, NR_{B}R_{C}, S(O₂)R_{A}, or optionally substituted alkyl, alkyl-aryl or alkyl-heterocycle;
R₃ is hydrogen, C(O)R_{A}, C(O)OR_{A}, or optionally substituted alkyl, alkyl-aryl, alkyl-heterocycle, aryl, or heterocycle;
R₄ is hydrogen or optionally substituted alkyl, alkyl-aryl, alkyl-heterocycle, aryl, or heterocycle;
each R_{A} is independently hydrogen or optionally substituted alkyl, alkyl-aryl or alkyl-heterocycle;
each R_{B} is independently hydrogen or optionally substituted alkyl, alkyl-aryl or alkyl-heterocycle;
each R_{C} is independently hydrogen or optionally substituted alkyl, alkyl-aryl or alkyl-heterocycle; and
m is 1-4.

5. The use of claim 4, wherein the compound is of the formula:

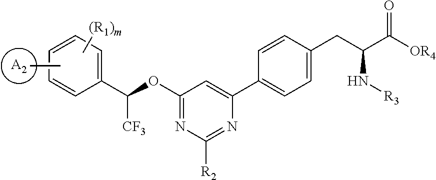

6. The use of claim 5, wherein the compound is of the formula: 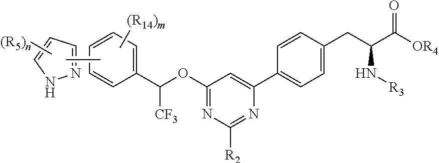
wherein:
each R₅ is independently halogen, hydrogen, C(O)R_{A}, OR_{A}, NR_{B}R_{C}, S(O₂)R_{A}, or optionally substituted alkyl, alkyl-aryl or alkyl-heterocycle; and
n is 1-3.

7. The use of claim 6, wherein the compound is of the formula: 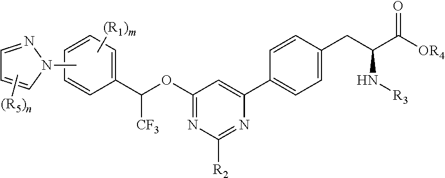
wherein:
each R₅ is independently halogen, hydrogen, C(O)R_{A}, OR_{A}, NR_{B}R_{C}, S(O₂)R_{A}, or optionally substituted alkyl, alkyl-aryl or alkyl-heterocycle; and
n is 1-3.

8. The use of claim 7, wherein R₁ is hydrogen or halogen.

9. The use of claim 7, wherein m is 1.

10. The use of claim 7, wherein R₂ is hydrogen or amino.

11. The use of claim 7, wherein R₄ is hydrogen or C₁₋₄ alkyl.

12. The use of claim 7, wherein R₅ is hydrogen or lower alkyl.

13. The use of claim 12, wherein R₅ is methyl.

14. The use of claim 1, wherein the compound is (S)-2-amino-3-(4-(2-amino-6-((R)-2,2,2-trifluoro-1-(3′-methoxybiphenyl-4-yl)ethoxy)pyrimidin-4-yl)phenyl)propanoic acid, or a pharmaceutically acceptable salt thereof.

15. The use of claim 1, wherein the compound is (S)-ethyl 2-amino-3-(4-(2-amino-6-((R)-1-(4-chloro-2-(3-methyl-1H-pyrazol-1-yl)phenyl)-2,2,2-trifluoroethoxy)pyrimidin-4-yl)phenyl)propanoate, or a pharmaceutically acceptable salt thereof.

16. The use of claim 1, wherein the compound is (S)-2-amino-3-(4-(2-amino-6-((R)-1-(4-chloro-2-(3-methyl-1H-pyrazol-1-yl)phenyl)-2,2,2-trifluoroethoxy)pyrimidin-4-yl)phenyl)propanoic acid, or a pharmaceutically acceptable salt thereof.
